SEQUENCE LISTING (235 sequences)

SEQ ID NO: 1 - protein, 9 aa, Artificial Sequence
  synthetic peptide
CDCRGDCFC

SEQ ID NO: 2 - protein, 13 aa, Artificial Sequence
  synthetic peptide
CGRECPRLCQSSC

SEQ ID NO: 3 - protein, 13 aa, Artificial Sequence
  synthetic peptide
CNGRCVSGCAGRC

SEQ ID NO: 4 - protein, 8 aa, Artificial Sequence
  synthetic peptide
CLSGSLSC

SEQ ID NO: 5 - protein, 7 aa, Artificial Sequence
  synthetic peptide
CGSLVRC

SEQ ID NO: 6 - protein, 6 aa, Artificial Sequence
  synthetic peptide
NGRAHA

SEQ ID NO: 7 - protein, 9 aa, Artificial Sequence
  synthetic peptide
CVLNGRMEC

SEQ ID NO: 8 - protein, 5 aa, Artificial Sequence
  synthetic peptide
CNGRC

SEQ ID NO: 9-11 - skipped (3 entries)

SEQ ID NO: 12 - protein, 13 aa, Artificial Sequence
  synthetic peptide
CXXXCXXXCXXXC

SEQ ID NO: 13 - protein, 9 aa, Artificial Sequence
  synthetic peptide
CXXXNGRXX

SEQ ID NO: 14 - protein, 9 aa, Artificial Sequence
  synthetic peptide
CXXCNGRCX

SEQ ID NO: 15 - protein, 12 aa, Artificial Sequence
  synthetic peptide
CNKTDGDEGVTC

SEQ ID NO: 16 - protein, 11 aa, Artificial Sequence
  synthetic peptide
ACDCRGDCFCG

SEQ ID NO: 17 - protein, 6 aa, Artificial Sequence
  synthetic peptide
GRGESP

SEQ ID NO: 18 - protein, 6 aa, Artificial Sequence
  synthetic peptide
WGTGLC

SEQ ID NO: 19 - protein, 13 aa, Artificial Sequence
  synthetic peptide
GACVFSIAHECGA

SEQ ID NO: 20 - protein, 13 aa, Artificial Sequence
  synthetic peptide
CGEACGGQCALPC

SEQ ID NO: 21 - protein, 9 aa, Artificial Sequence
  synthetic peptide
IWSGYGVYW

SEQ ID NO: 22 - protein, 9 aa, Artificial Sequence
  synthetic peptide
PSCAYMCIT

SEQ ID NO: 23 - protein, 9 aa, Artificial Sequence
  synthetic peptide
WESLYFPRE

SEQ ID NO: 24 - protein, 9 aa, Artificial Sequence
  synthetic peptide
SKVLYYNWE

SEQ ID NO: 25 - protein, 13 aa, Artificial Sequence
  synthetic peptide
CGLMCQGACFDVC

SEQ ID NO: 26 - protein, 13 aa, Artificial Sequence
  synthetic peptide
CERACRNLCREGC

SEQ ID NO: 27 - protein, 13 aa, Artificial Sequence
  synthetic peptide
CPRGCLAVCVSQC

SEQ ID NO: 28 - protein, 10 aa, Artificial Sequence
  synthetic peptide
CKVCNGRCCG

SEQ ID NO: 29 - protein, 10 aa, Artificial Sequence
  synthetic peptide
CEMCNGRCMG

SEQ ID NO: 30 - protein, 10 aa, Artificial Sequence
  synthetic peptide
CPLCNGRCAL

SEQ ID NO: 31 - protein, 10 aa, Artificial Sequence
  synthetic peptide
CPTCNGRCVR

SEQ ID NO: 32 - protein, 10 aa, Artificial Sequence
  synthetic peptide
CGVCNGRCGL

SEQ ID NO: 33 - protein, 10 aa, Artificial Sequence
  synthetic peptide
CEQCNGRCGQ

SEQ ID NO: 34 - protein, 10 aa, Artificial Sequence
  synthetic peptide
CRNCNGRCEG

SEQ ID NO: 35 - protein, 10 aa, Artificial Sequence
  synthetic peptide
CVLCNGRCWS

SEQ ID NO: 36 - protein, 10 aa, Artificial Sequence
  synthetic peptide
CVTCNGRCRV

SEQ ID NO: 37 - protein, 10 aa, Artificial Sequence
  synthetic peptide
CTECNGRCQL

SEQ ID NO: 38 - protein, 10 aa, Artificial Sequence
  synthetic peptide
CRTCNGRCLE

SEQ ID NO: 39 - protein, 10 aa, Artificial Sequence
  synthetic peptide
CETCNGRCVG

SEQ ID NO: 40 - protein, 10 aa, Artificial Sequence
  synthetic peptide
CAVCNGRCGF

SEQ ID NO: 41 - protein, 10 aa, Artificial Sequence
  synthetic peptide
CRDLNGRKVM

SEQ ID NO: 42 - protein, 10 aa, Artificial Sequence
  synthetic peptide
CSCCNGRCGD

SEQ ID NO: 43 - protein, 10 aa, Artificial Sequence
  synthetic peptide
CWGCNGRCRM

SEQ ID NO: 44 - protein, 10 aa, Artificial Sequence
  synthetic peptide
CPLCNGRCAR

SEQ ID NO: 45 - protein, 10 aa, Artificial Sequence
  synthetic peptide
CKSCNGRCLA

SEQ ID NO: 46 - protein, 10 aa, Artificial Sequence
  synthetic peptide
CVPCNGRCHE

SEQ ID NO: 47 - protein, 10 aa, Artificial Sequence
  synthetic peptide
CQSCNGRCVR

SEQ ID NO: 48 - protein, 10 aa, Artificial Sequence
  synthetic peptide
CRTCNGRCQV

SEQ ID NO: 49 - protein, 10 aa, Artificial Sequence
  synthetic peptide
CVQCNGRCAL

SEQ ID NO: 50 - protein, 9 aa, Artificial Sequence
  synthetic peptide
CRCCNGRCS

SEQ ID NO: 51 - protein, 10 aa, Artificial Sequence
  synthetic peptide
CASNNGRVVL

SEQ ID NO: 52 - protein, 10 aa, Artificial Sequence
  synthetic peptide
CGRCNGRCLL

SEQ ID NO: 53 - protein, 10 aa, Artificial Sequence
  synthetic peptide
CWLCNGRCGR

SEQ ID NO: 54 - protein, 10 aa, Artificial Sequence
  synthetic peptide
CSKCNGRCGH

SEQ ID NO: 55 - protein, 10 aa, Artificial Sequence
  synthetic peptide
CVWCNGRCGL

SEQ ID NO: 56 - protein, 10 aa, Artificial Sequence
  synthetic peptide
CIRCNGRCSV

SEQ ID NO: 57 - protein, 10 aa, Artificial Sequence
  synthetic peptide
CGECNGRCVE

SEQ ID NO: 58 - protein, 10 aa, Artificial Sequence
  synthetic peptide
CEGVNGRRLR

SEQ ID NO: 59 - protein, 10 aa, Artificial Sequence
  synthetic peptide
CLSCNGRCPS

SEQ ID NO: 60 - protein, 10 aa, Artificial Sequence
  synthetic peptide
CEVCNGRCAL

SEQ ID NO: 61 - protein, 7 aa, Artificial Sequence
  synthetic peptide
GRSQMQI

SEQ ID NO: 62 - protein, 7 aa, Artificial Sequence
  synthetic peptide
HHTRFVS

SEQ ID NO: 63 - protein, 7 aa, Artificial Sequence
  synthetic peptide
SKGLRHR

SEQ ID NO: 64 - protein, 7 aa, Artificial Sequence
  synthetic peptide
VASVSVA

SEQ ID NO: 65 - protein, 7 aa, Artificial Sequence
  synthetic peptide
WRVLAAF

SEQ ID NO: 66 - protein, 7 aa, Artificial Sequence
  synthetic peptide
KMGPKVW

SEQ ID NO: 67 - protein, 7 aa, Artificial Sequence
  synthetic peptide
IFSGSRE

SEQ ID NO: 68 - protein, 7 aa, Artificial Sequence
  synthetic peptide
SPGSWTW

SEQ ID NO: 69 - protein, 7 aa, Artificial Sequence
  synthetic peptide
NPRWFWD

SEQ ID NO: 70 - protein, 7 aa, Artificial Sequence
  synthetic peptide
GRWYKWA

SEQ ID NO: 71 - protein, 7 aa, Artificial Sequence
  synthetic peptide
IKARASP

SEQ ID NO: 72 - protein, 7 aa, Artificial Sequence
  synthetic peptide
SGWCYRC

SEQ ID NO: 73 - protein, 7 aa, Artificial Sequence
  synthetic peptide
ALVGLMR

SEQ ID NO: 74 - protein, 7 aa, Artificial Sequence
  synthetic peptide
LWAEMTG

SEQ ID NO: 75 - protein, 7 aa, Artificial Sequence
  synthetic peptide
CWSGVDC

SEQ ID NO: 76 - protein, 7 aa, Artificial Sequence
  synthetic peptide
DTLRLRI

SEQ ID NO: 77 - protein, 7 aa, Artificial Sequence
  synthetic peptide
SKSSGVS

SEQ ID NO: 78 - protein, 7 aa, Artificial Sequence
  synthetic peptide
IVADYQR

SEQ ID NO: 79 - protein, 7 aa, Artificial Sequence
  synthetic peptide
VWRTGHL

SEQ ID NO: 80 - protein, 7 aa, Artificial Sequence
  synthetic peptide
VVDRFPD

SEQ ID NO: 81 - protein, 7 aa, Artificial Sequence
  synthetic peptide
LSMFTRP

SEQ ID NO: 82 - protein, 7 aa, Artificial Sequence
  synthetic peptide
GLPVKWS

SEQ ID NO: 83 - protein, 7 aa, Artificial Sequence
  synthetic peptide
IMYPGWL

SEQ ID NO: 84 - protein, 9 aa, Artificial Sequence
  synthetic peptide
CVMVRDGDC

SEQ ID NO: 85 - protein, 7 aa, Artificial Sequence
  synthetic peptide
CVRIRPC

SEQ ID NO: 86 - protein, 7 aa, Artificial Sequence
  synthetic peptide
CQLAAVC

SEQ ID NO: 87 - protein, 7 aa, Artificial Sequence
  synthetic peptide
CGVGSSC

SEQ ID NO: 88 - protein, 7 aa, Artificial Sequence
  synthetic peptide
CVSGPRC

SEQ ID NO: 89 - protein, 7 aa, Artificial Sequence
  synthetic peptide
CGLSDSC

SEQ ID NO: 90 - protein, 7 aa, Artificial Sequence
  synthetic peptide
CGEGHPC

SEQ ID NO: 91 - protein, 7 aa, Artificial Sequence
  synthetic peptide
CYTADPC

SEQ ID NO: 92 - protein, 9 aa, Artificial Sequence
  synthetic peptide
CELSLISKC

SEQ ID NO: 93 - protein, 9 aa, Artificial Sequence
  synthetic peptide
CPEHRSLVC

SEQ ID NO: 94 - protein, 9 aa, Artificial Sequence
  synthetic peptide
CLVVHEAAC

SEQ ID NO: 95 - protein, 7 aa, Artificial Sequence
  synthetic peptide
CYVELHC

SEQ ID NO: 96 - protein, 7 aa, Artificial Sequence
  synthetic peptide
CWRKFYC

SEQ ID NO: 97 - protein, 7 aa, Artificial Sequence
  synthetic peptide
CFWPNRC

SEQ ID NO: 98 - protein, 8 aa, Artificial Sequence
  synthetic peptide
CYSYFLAC

SEQ ID NO: 99 - protein, 7 aa, Artificial Sequence
  synthetic peptide
CPRGSRC

SEQ ID NO: 100 - protein, 7 aa, Artificial Sequence
  synthetic peptide
CRLGIAC

SEQ ID NO: 101 - protein, 8 aa, Artificial Sequence
  synthetic peptide
CDDSWKCP

SEQ ID NO: 102 - protein, 9 aa, Artificial Sequence
  synthetic peptide
CAQLLQVSC

SEQ ID NO: 103 - protein, 7 aa, Artificial Sequence
  synthetic peptide
CYPADPC

SEQ ID NO: 104 - protein, 8 aa, Artificial Sequence
  synthetic peptide
CKALSQAC

SEQ ID NO: 105 - protein, 7 aa, Artificial Sequence
  synthetic peptide
CTDYVRC

SEQ ID NO: 106 - protein, 7 aa, Artificial Sequence
  synthetic peptide
CGETMRC

SEQ ID NO: 107 - protein, 9 aa, Artificial Sequence
  synthetic peptide
GICKDDWCQ

SEQ ID NO: 108 - protein, 9 aa, Artificial Sequence
  synthetic peptide
TSCDPSLCE

SEQ ID NO: 109 - protein, 9 aa, Artificial Sequence
  synthetic peptide
KGCGTRQCW

SEQ ID NO: 110 - protein, 9 aa, Artificial Sequence
  synthetic peptide
YRCREVLCQ

SEQ ID NO: 111 - protein, 7 aa, Artificial Sequence
  synthetic peptide
CWGTGLC

SEQ ID NO: 112 - protein, 9 aa, Artificial Sequence
  synthetic peptide
WSCADRTCM

SEQ ID NO: 113 - protein, 9 aa, Artificial Sequence
  synthetic peptide
AGCRLKSCA

SEQ ID NO: 114 - protein, 9 aa, Artificial Sequence
  synthetic peptide
SRCKTGLCQ

SEQ ID NO: 115 - protein, 9 aa, Artificial Sequence
  synthetic peptide
PICEVSRCW

SEQ ID NO: 116 - protein, 9 aa, Artificial Sequence
  synthetic peptide
WTCRASWCS

SEQ ID NO: 117 - protein, 9 aa, Artificial Sequence
  synthetic peptide
GRCLLMQCR

SEQ ID NO: 118 - protein, 9 aa, Artificial Sequence
  synthetic peptide
TECDMSRCM

SEQ ID NO: 119 - protein, 9 aa, Artificial Sequence
  synthetic peptide
ARCRVDPCV

SEQ ID NO: 120 - protein, 9 aa, Artificial Sequence
  synthetic peptide
CIEGVLGGC

SEQ ID NO: 121 - protein, 7 aa, Artificial Sequence
  synthetic peptide
CSVANSC

SEQ ID NO: 122 - protein, 7 aa, Artificial Sequence
  synthetic peptide
CSSTMRC

SEQ ID NO: 123 - protein, 7 aa, Artificial Sequence
  synthetic peptide
SIDSTTF

SEQ ID NO: 124 - protein, 7 aa, Artificial Sequence
  synthetic peptide
GPSRVGG

SEQ ID NO: 125 - protein, 7 aa, Artificial Sequence
  synthetic peptide
WWSGLEA

SEQ ID NO: 126 - protein, 7 aa, Artificial Sequence
  synthetic peptide
LGTDVRQ

SEQ ID NO: 127 - protein, 7 aa, Artificial Sequence
  synthetic peptide
LVGVRLL

SEQ ID NO: 128 - protein, 7 aa, Artificial Sequence
  synthetic peptide
GRPGDIW

SEQ ID NO: 129 - protein, 7 aa, Artificial Sequence
  synthetic peptide
TVWNPVG

SEQ ID NO: 130 - protein, 7 aa, Artificial Sequence
  synthetic peptide
GLLLVVP

SEQ ID NO: 131 - protein, 7 aa, Artificial Sequence
  synthetic peptide
FAATSAE

SEQ ID NO: 132 - protein, 7 aa, Artificial Sequence
  synthetic peptide
WCCRQFN

SEQ ID NO: 133 - protein, 7 aa, Artificial Sequence
  synthetic peptide
VGFGKAL

SEQ ID NO: 134 - protein, 7 aa, Artificial Sequence
  synthetic peptide
DSSLRLP

SEQ ID NO: 135 - protein, 7 aa, Artificial Sequence
  synthetic peptide
KLWCAMS

SEQ ID NO: 136 - protein, 7 aa, Artificial Sequence
  synthetic peptide
SLVSFLG

SEQ ID NO: 137 - protein, 7 aa, Artificial Sequence
  synthetic peptide
GSFAFLV

SEQ ID NO: 138 - protein, 7 aa, Artificial Sequence
  synthetic peptide
IASVRWA

SEQ ID NO: 139 - protein, 7 aa, Artificial Sequence
  synthetic peptide
TWGHLRA

SEQ ID NO: 140 - protein, 7 aa, Artificial Sequence
  synthetic peptide
QYREGLV

SEQ ID NO: 141 - protein, 7 aa, Artificial Sequence
  synthetic peptide
QSADRSV

SEQ ID NO: 142 - protein, 7 aa, Artificial Sequence
  synthetic peptide
YMFWTSR

SEQ ID NO: 143 - protein, 7 aa, Artificial Sequence
  synthetic peptide
LVRRWYL

SEQ ID NO: 144 - protein, 7 aa, Artificial Sequence
  synthetic peptide
TARGSSR

SEQ ID NO: 145 - protein, 7 aa, Artificial Sequence
  synthetic peptide
TTREKNL

SEQ ID NO: 146 - protein, 7 aa, Artificial Sequence
  synthetic peptide
PKWLLFS

SEQ ID NO: 147 - protein, 7 aa, Artificial Sequence
  synthetic peptide
LRTNVVH

SEQ ID NO: 148 - protein, 7 aa, Artificial Sequence
  synthetic peptide
AVMGLAA

SEQ ID NO: 149 - protein, 7 aa, Artificial Sequence
  synthetic peptide
VRNSLRN

SEQ ID NO: 150 - protein, 9 aa, Artificial Sequence
  synthetic peptide
TDCTPSRCT

SEQ ID NO: 151 - protein, 9 aa, Artificial Sequence
  synthetic peptide
SWCQFEKCL

SEQ ID NO: 152 - protein, 9 aa, Artificial Sequence
  synthetic peptide
VPCRFKQCW

SEQ ID NO: 153 - protein, 9 aa, Artificial Sequence
  synthetic peptide
CTAMRNTDC

SEQ ID NO: 154 - protein, 8 aa, Artificial Sequence
  synthetic peptide
CRESLKNC

SEQ ID NO: 155 - protein, 8 aa, Artificial Sequence
  synthetic peptide
CMEMGVKC

SEQ ID NO: 156 - protein, 9 aa, Artificial Sequence
  synthetic peptide
VTCRSLMCQ

SEQ ID NO: 157 - protein, 8 aa, Artificial Sequence
  synthetic peptide
CNNVGSYC

SEQ ID NO: 158 - protein, 8 aa, Artificial Sequence
  synthetic peptide
CGTRVDHC

SEQ ID NO: 159 - protein, 8 aa, Artificial Sequence
  synthetic peptide
CISLDRSC

SEQ ID NO: 160 - protein, 8 aa, Artificial Sequence
  synthetic peptide
CAMVSMED

SEQ ID NO: 161 - protein, 8 aa, Artificial Sequence
  synthetic peptide
CYLGVSNC

SEQ ID NO: 162 - protein, 8 aa, Artificial Sequence
  synthetic peptide
CYLVNVDC

SEQ ID NO: 163 - protein, 8 aa, Artificial Sequence
  synthetic peptide
CIRSAVSC

SEQ ID NO: 164 - protein, 9 aa, Artificial Sequence
  synthetic peptide
LVCLPPSCE

SEQ ID NO: 165 - protein, 9 aa, Artificial Sequence
  synthetic peptide
RHCFSQWCS

SEQ ID NO: 166 - protein, 9 aa, Artificial Sequence
  synthetic peptide
FYCPGVGCR

SEQ ID NO: 167 - protein, 9 aa, Artificial Sequence
  synthetic peptide
ISCAVDACL

SEQ ID NO: 168 - protein, 9 aa, Artificial Sequence
  synthetic peptide
EACEMAGCL

SEQ ID NO: 169 - protein, 9 aa, Artificial Sequence
  synthetic peptide
PRCESQLCP

SEQ ID NO: 170 - protein, 9 aa, Artificial Sequence
  synthetic peptide
RSCIKHQCP

SEQ ID NO: 171 - protein, 9 aa, Artificial Sequence
  synthetic peptide
QWCSRRWCT

SEQ ID NO: 172 - protein, 9 aa, Artificial Sequence
  synthetic peptide
MFCRMRSCD

SEQ ID NO: 173 - protein, 9 aa, Artificial Sequence
  synthetic peptide
GICKDLWCQ

SEQ ID NO: 174 - protein, 9 aa, Artificial Sequence
  synthetic peptide
NACESAICG

SEQ ID NO: 175 - protein, 9 aa, Artificial Sequence
  synthetic peptide
APCGLLACI

SEQ ID NO: 176 - protein, 9 aa, Artificial Sequence
  synthetic peptide
NRCRGVSCT

SEQ ID NO: 177 - protein, 9 aa, Artificial Sequence
  synthetic peptide
FPCEGKKCL

SEQ ID NO: 178 - protein, 9 aa, Artificial Sequence
  synthetic peptide
ADCRQKPCL

SEQ ID NO: 179 - protein, 9 aa, Artificial Sequence
  synthetic peptide
FGCVMASCR

SEQ ID NO: 180 - protein, 9 aa, Artificial Sequence
  synthetic peptide
AGCINGLCG

SEQ ID NO: 181 - protein, 9 aa, Artificial Sequence
  synthetic peptide
RSCAEPWCY

SEQ ID NO: 182 - protein, 9 aa, Artificial Sequence
  synthetic peptide
DTCRALRCN

SEQ ID NO: 183 - protein, 9 aa, Artificial Sequence
  synthetic peptide
GRCVDGGCT

SEQ ID NO: 184 - protein, 9 aa, Artificial Sequence
  synthetic peptide
YRCIARECE

SEQ ID NO: 185 - protein, 9 aa, Artificial Sequence
  synthetic peptide
KRCSSSLCA

SEQ ID NO: 186 - protein, 8 aa, Artificial Sequence
  synthetic peptide
ICLLAHCA

SEQ ID NO: 187 - protein, 9 aa, Artificial Sequence
  synthetic peptide
QACPMLLCM

SEQ ID NO: 188 - protein, 9 aa, Artificial Sequence
  synthetic peptide
LDCLSELCS

SEQ ID NO: 189 - protein, 8 aa, Artificial Sequence
  synthetic peptide
AGCRVESC

SEQ ID NO: 190 - protein, 9 aa, Artificial Sequence
  synthetic peptide
HTCLVALCA

SEQ ID NO: 191 - protein, 9 aa, Artificial Sequence
  synthetic peptide
IYCPGQECE

SEQ ID NO: 192 - protein, 9 aa, Artificial Sequence
  synthetic peptide
RLCSLYGCV

SEQ ID NO: 193 - protein, 9 aa, Artificial Sequence
  synthetic peptide
RKCEVPGCQ

SEQ ID NO: 194 - protein, 9 aa, Artificial Sequence
  synthetic peptide
EDCTSRFCS

SEQ ID NO: 195 - protein, 9 aa, Artificial Sequence
  synthetic peptide
LECVVDSCR

SEQ ID NO: 196 - protein, 9 aa, Artificial Sequence
  synthetic peptide
EICVDGLCV

SEQ ID NO: 197 - protein, 9 aa, Artificial Sequence
  synthetic peptide
RWCREKSCW

SEQ ID NO: 198 - protein, 9 aa, Artificial Sequence
  synthetic peptide
FRCLERVCT

SEQ ID NO: 199 - protein, 9 aa, Artificial Sequence
  synthetic peptide
RPCGDQACE

SEQ ID NO: 200 - protein, 14 aa, Artificial Sequence
  synthetic peptide
KLAKLAKKLAKLAK

SEQ ID NO: 201 - protein, 14 aa, Artificial Sequence
  synthetic peptide
KLAKKLAKLAKKLA

SEQ ID NO: 202 - protein, 14 aa, Artificial Sequence
  synthetic peptide
KAAKKAAKAAKKAA

SEQ ID NO: 203 - protein, 21 aa, Artificial Sequence
  synthetic peptide
KLGKKLGKLGKKLGKLGKKLG

SEQ ID NO: 204 - protein, 14 aa, Artificial Sequence
  synthetic peptide
DLSLARLATARLAI

SEQ ID NO: 205-206 - skipped (2 entries)

SEQ ID NO: 207 - protein, 7 aa, Artificial Sequence
  synthetic peptide
SMSIARL

SEQ ID NO: 208 - protein, 5 aa, Artificial Sequence
  synthetic peptide
CARAC

SEQ ID NO: 209 - protein, 7 aa, Artificial Sequence
  synthetic peptide
EVQSAKW

SEQ ID NO: 210 - protein, 7 aa, Artificial Sequence
  synthetic peptide
KRVYVLG

SEQ ID NO: 211 - protein, 7 aa, Artificial Sequence
  synthetic peptide
GRLSVQV

SEQ ID NO: 212 - protein, 7 aa, Artificial Sequence
  synthetic peptide
WKPASLS

SEQ ID NO: 213 - protein, 7 aa, Artificial Sequence
  synthetic peptide
FAVRVVG

SEQ ID NO: 214 - protein, 7 aa, Artificial Sequence
  synthetic peptide
LVRPLEG

SEQ ID NO: 215 - protein, 7 aa, Artificial Sequence
  synthetic peptide
GFYRMLG

SEQ ID NO: 216 - protein, 7 aa, Artificial Sequence
  synthetic peptide
EGRPMVY

SEQ ID NO: 217 - protein, 7 aa, Artificial Sequence
  synthetic peptide
GSRSLGA

SEQ ID NO: 218 - protein, 7 aa, Artificial Sequence
  synthetic peptide
RVWQGDV

SEQ ID NO: 219 - protein, 6 aa, Artificial Sequence
  synthetic peptide
GDELLA

SEQ ID NO: 220 - protein, 7 aa, Artificial Sequence
  synthetic peptide
FVWLVGS

SEQ ID NO: 221 - protein, 7 aa, Artificial Sequence
  synthetic peptide
GSEPMFR

SEQ ID NO: 222 - protein, 7 aa, Artificial Sequence
  synthetic peptide
VSFLEYR

SEQ ID NO: 223 - protein, 5 aa, Artificial Sequence
  synthetic peptide
WHQPL

SEQ ID NO: 224 - protein, 7 aa, Artificial Sequence
  synthetic peptide
RGRWLAL

SEQ ID NO: 225 - protein, 7 aa, Artificial Sequence
  synthetic peptide
QVEEFPC

SEQ ID NO: 226 - protein, 7 aa, Artificial Sequence
  synthetic peptide
LWLSGNW

SEQ ID NO: 227 - protein, 7 aa, Artificial Sequence
  synthetic peptide
GPMLSVM

SEQ ID NO: 228 - protein, 7 aa, Artificial Sequence
  synthetic peptide
WTFLERL

SEQ ID NO: 229 - protein, 7 aa, Artificial Sequence
  synthetic peptide
VLPGGQW

SEQ ID NO: 230 - protein, 6 aa, Artificial Sequence
  synthetic peptide
REVKES

SEQ ID NO: 231 - protein, 7 aa, Artificial Sequence
  synthetic peptide
RTPAAVM

SEQ ID NO: 232 - protein, 7 aa, Artificial Sequence
  synthetic peptide
GEWLGEC

SEQ ID NO: 233 - protein, 7 aa, Artificial Sequence
  synthetic peptide
PNPLMPL

SEQ ID NO: 234 - protein, 7 aa, Artificial Sequence
  synthetic peptide
SLWYLGA

SEQ ID NO: 235 - protein, 7 aa, Artificial Sequence
  synthetic peptide
YVGGWEL

(19) United States
(12) Patent Application Publication
(10) Pub. No.: US 2001/0046498 A1
(43) Pub. Date: Nov. 29, 2001

(54) Chimeric prostate-homing peptides with pro-apoptotic activity

(76) Inventors: Erkki I. Ruoslahti, Rancho Santa Fe, CA (US); Renata Pasqualini, Houston, TX (US); Wadih Arap, Houston, TX (US); Dale E. Bredesen, Novato, CA (US); H. Michael Ellerby, Novato, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 09/765,086

(22) Filed: Jan. 17, 2001

Related U.S. Application Data
(60) Provisional application No. 60/266,317, filed on Jan. 21, 2000.

(51) Int. Cl. A61K 39/395; C07K 16/30

(52) U.S. Cl. 424/178.1; 530/391.7

(57) ABSTRACT

The present invention provides a chimeric prostate-homing peptide with pro-apoptotic activity. In a preferred embodiment, the chimeric prostate-homing pro-apoptotic peptide contains the sequence SMSIARL-GG-_{D}(KLAKLAK)₂. Methods of using such chimeric peptides for treating patients having prostate cancer also are provided.

This application is based on, and claims the benefit of, U.S. Provisional Application No. ______ (yet to be assigned), filed Jan. 21, 2000, which was converted from U.S. Ser. No. 09/489,582, and entitled CHIMERIC PROSTATE-HOMING PEPTIDES WITH PRO-APOPTOTIC ACTIVITY, and which is incorporated herein by reference.

This work was supported by grants CA74238, CA28896 and CA30199 from the National Cancer Institute (USA), and by grant DAMD17-98-1-8581 from the Department of Defense. The United States government has certain rights in this invention.

### BACKGROUND OF THE INVENTION

1. Field of the Invention

The present invention relates generally to the fields of cancer biology and drug delivery and, more specifically, to the selective targeting of antimicrobial peptides to the prostate.

2. Background Information

Prostatic adenocarcinoma recently became the most frequent cancer diagnosed in American men, surpassing the frequency of lung cancer for the first time. An estimated 30,000 American men die annually of this malignancy. Furthermore, prostate cancer affects men throughout the world, with the highest frequencies reported in the United States and Scandinavian countries and a moderately high frequency seen throughout Europe. Prostate cancer is a disease of elderly men, and of all patients with this disease, 75% are 60 to 80 years of age. At the age of 50 years, the estimated lifetime probability of developing clinically apparent prostatic carcinoma is about 10% for American men. However, autopsy studies have shown that the true frequency of prostatic carcinoma is actually considerably higher than is indicated by clinical evidence.

A major hurdle to advances in treating cancers such as cancer of the prostate is the relative lack of agents that can selectively target the cancer, while sparing normal tissue. For example, radiation therapy and surgery, which generally are localized treatments, can cause substantial damage to normal tissue in the treatment field, resulting in scarring and, in severe cases, loss of function of the normal tissue. Chemotherapy, which generally is administered systemically, can cause substantial damage to organs such as bone marrow, mucosae, skin and the small intestine, which undergo rapid cell turnover and continuous cell division. As a result, undesirable side effects, for example, nausea, hair loss and reduced blood cell counts, occur as a result of systemically treating a cancer patient with chemotherapeutic agents. Such undesirable side effects often limit the amount of a treatment that can be administered. Due to such shortcomings in treatment, cancer remains a leading cause of patient morbidity and death.

Potent antimicrobial activity has been observed for a class of peptides including naturally occurring peptides such as melittin, the gramicidins, magainins, defensins and cecropins. Naturally occurring antimicrobial peptides, and related synthetic antimicrobial sequences, generally have an equivalent number of polar and nonpolar residues within an amphipathic domain and a sufficient number of basic residues to give the peptide an overall positive charge at neutral pH. The biological activity of amphipathic α-helical peptides against Gram-positive bacteria may result from the ability of these peptides to form ion channels through membrane bilayers. Many antimicrobial peptides selectively inhibit and kill bacteria while maintaining low mammalian cell cytotoxicity, with the differential sensitivity of bacterial cells apparently due to membrane differences between bacteria and mammalian cells. As shown herein, these antimicrobial peptides can be endowed with selective cytotoxic activity against a particular eukaryotic cell type, such as the endothelium of the prostate gland.

Thus, there is a need for novel anti-cancer therapeutics that can selectively target the prostate. The present invention satisfies this need by providing prostate-homing pro-apoptotic peptides that combine an antimicrobial peptide with a prostate-homing peptide to produce a conjugate with selective toxicity against the prostate. Related advantages are provided as well.

### SUMMARY OF THE INVENTION

The present invention provides a chimeric prostate-homing pro-apoptotic peptide that contains a prostate-homing peptide linked to an antimicrobial peptide, where the chimeric peptide is selectively internalized by prostate tissue and exhibits high toxicity thereto, while the antimicrobial peptide has low mammalian cell toxicity when not linked to the prostate-homing peptide. In a chimeric peptide of the invention, the prostate-homing peptide portion can contain, for example, the sequence SMSIARL (SEQ ID NO: 207) or a functionally equivalent sequence, and the antimicrobial peptide portion can have an amphipathic α-helical structure such as the sequence (KLAKLAK)₂ (SEQ ID NO: 200), (KLAKKLA)₂ (SEQ ID NO: 201), (KAAKKAA)₂ (SEQ ID NO: 202) or (KLGKKLG)₃ (SEQ ID NO: 203). In a preferred embodiment, the antimicrobial peptide portion contains the sequence _{D}(KLAKLAK)₂. An exemplary prostate-homing pro-apoptotic peptide is provided herein as SMSIARL-GG-_{D}(KLAKLAK)₂. The present invention further provides a method of directing an antimicrobial peptide to a prostate cancer in vivo. The method includes the step of administering a chimeric prostate-homing pro-apoptotic peptide that contains a prostate-homing peptide linked to an antimicrobial peptide, where the chimeric peptide is selectively internalized by prostate tissue and exhibits high toxicity thereto, while the antimicrobial peptide has low mammalian cell toxicity when not linked to the prostate-homing peptide. In a method of the invention, the prostate-homing peptide can contain, for example, the sequence SMSIARL (SEQ ID NO: 207) or a functionally equivalent sequence, and the antimicrobial peptide can contain a sequence such as _{D}(KLAKLAK) ₂. In a preferred embodiment, the chimeric prostate-homing pro-apoptotic peptide includes the sequence SMSIARL-GG-_{D}(KLAKLAK)₂.

Also provided by the invention is a method of inducing selective toxicity in a prostate cancer in vivo. The method includes the step of administering to a subject having prostate cancer a chimeric prostate-homing pro-apoptotic peptide that contains a prostate-homing peptide linked to an antimicrobial peptide, where the chimeric peptide is selectively internalized by prostate tissue and exhibits high toxicity thereto, while the antimicrobial peptide has low mammalian cell toxicity when not linked to the prostate-homing peptide. The method of inducing selective toxicity in a prostate cancer in vivo can be practiced, for example, with a prostate-homing peptide containing the sequence SMSIARL (SEQ ID NO: 207) or a functionally equivalent sequence. The antimicrobial peptide can include, for example, the sequence _{D}(KLAKLAK)₂. In a preferred embodiment, the chimeric prostate-homing pro-apoptotic peptide includes the sequence SMSIARL-GG-_{D}(KLAKLAK)₂. In addition, the invention provides a method of treating a patient having prostate cancer by administering to the patient a chimeric prostate-homing pro-apoptotic peptide of the invention, whereby the chimeric peptide is selectively toxic to the tumor. The chimeric peptide contains a prostate-homing peptide linked to an antimicrobial peptide, and the chimeric peptide is selectively internalized by prostate tissue and exhibits high toxicity thereto, while the antimicrobial peptide has low mammalian cell toxicity when not linked to the prostate-homing peptide. The prostate-homing peptide portion can contain, for example, the sequence SMSIARL (SEQ ID NO: 207) or a functionally equivalent sequence, and the antimicrobial peptide portion can contain, for example, the sequence _{D}(KLAKLAK)₂. In a preferred embodiment, the chimeric peptide contains the sequence SMSIARL-GG-_{D}(KLAKLAK)₂.

### BRIEF DESCRIPTION OF THE DRAWINGS

FIG. 1 shows a computer-generated model and amino acid sequence of CNGRC-GG-_{D}(KLAKLAK)₂, designated “HPP-1.”

Upper panel: CNGRC-GG-_{D}(KLAKLAK)₂ (HPP-1) is composed of a homing domain and a membrane-disrupting domain joined by a coupling domain.

Lower panel: Amino acid sequence of “HPP-1” corresponding to the structure shown in upper panel.

FIG. 2 shows mitochondrial swelling and mitochondria-dependent apoptosis in the presence of _{D}(KLAKLAK)₂.

a. Mitochondrial swelling curve (optical absorbance spectrum) is shown in the presence of _{D}(KLAKLAK)₂ or CA⁺² (positive control).

b. Immunoblot of caspase-3 cleavage showing the 32 kDa proform and 8 and 20 kDa processed forms in the presence of _{D}(KLAKLAK)₂ or DLSLARLATARLAI (SEQ ID NO: 204) in the presence or absence of mitochondria. Typical experiments are shown. Results were reproduced in three independent experiments.

FIG. 3 shows mitochondrial swelling and apoptosis in dermal microvessel endothelial cells treated with CNGRC-GG-_{D}(KLAKLAK)₂ (HPP-1).

a. Dermal microvessel endothelial cell cord formation, scale bar=250 μm.

b. DEVD-pNA hydrolysis (caspase activation) in proliferating dermal microvessel endothelial cells treated with CNGRC-GG-_{D}(KLAKLAK)₂ (HPP-1).

c. Viability of proliferating dermal microvessel endothelial cells treated with HPP-1 (black bars) or control peptide _{D}(KLAKLAK)₂ (gray bars) over time. (t test, P<0.05).

d. Viability of cord-forming dermal microvessel endothelial cells treated with HPP-1 (black bars) or control peptide _{D}(KLAKLAK)₂ (gray bars) over time. (t test, P<0.05).

FIG. 4 shows the effect of HPP-1 treatment of nude mice bearing human MDA-MB-435-derived breast carcinoma xenografts.

a. Tumor volume of HPP-1 treated tumors as compared to control CARAC-GG-_{D}(KLAKLAK)₂ treated tumors. Differences in tumor volumes between day 1 and day 50 are shown (t test, P=0.027).

b. Kaplan-Meier survival plot showing the survival of nude mice bearing human MDA-MB-435-derived breast carcinoma xenografts treated with HPP-1 or control peptide (mixture of _{D}(KLAKLAK)₂ and CNGRC (SEQ ID NO: 8). Each group was comprised of thirteen animals. (Log-Rank Test, P<0.05).

FIG. 5 shows the effect of CDCRGDCFC-GG-_{D}(KLAKLAK)₂ on oxygen-induced retinal neovascularization in newborn mice. Retinal neovessel number is shown for treatments with vehicle (black bar); CDCRGDCFC-GG-_{D}(KLAKLAK)₂ (striped bar); and a control mixture of unconjugated CDCRGDCFC (SEQ ID NO: 1) and _{D}(KLAKLAK)₂ (hatched bar).

FIG. 6 shows accumulation of intravenously injected biotin conjugate of prostate-homing peptide in prostate tissue. a. Avidin-peroxidase staining of a prostate section from a mouse injected with biotin-labeled prostate homing peptide, SMSIARL (SEQ ID NO: 207). b. Avidin-peroxidase staining of a prostate section from a mouse injected with biotin-labeled control peptide CARAC (SEQ ID NO: 208).

FIG. 7 shows apoptosis induced by SMSIARL-GG-_{D}(KLAKLAK)₂ in the normal mouse prostate. a. TUNEL staining of prostate tissue from a mouse treated with SMSIARL-GG-_{D}(KLAKLAK)₂ chimeric peptide. b. Larger magnification of a field similar to that in a. c. TUNEL staining of negative control mice treated with 250 μg of an unconjugated mixture of SMSIARL (SEQ ID NO: 207) and _{D}(KLAKLAK)₂ .

FIG. 8 shows survival of TRAMP mice treated with SMSIARL-GG-_{D}(KLAKLAK)₂, vehicle alone, _{D}(KLAKLAK)₂ peptide alone, or SMSIARL peptide (SEQ ID NO: 207) alone.

FIG. 9 shows binding of prostate-homing SMSIARL (SEQ ID NO: 207) phage to human prostate vasculature. a and b. Peroxidase staining of human prostate tissue section containing both normal and cancerous tissue overlaid with 10⁹ TU SMSIARL phage (SEQ ID NO: 207) and detected with anti-phage antibody. a is an overview (×20) while b shows a detail from panel a at a higher magnification (×40). c. Peroxidase staining as in panel a with phage lacking a peptide insert. d. Peroxidase staining as in a with soluble SMSIARL peptide SEQ ID NO: 207 included in the overlay.

### DETAILED DESCRIPTION OF THE INVENTION

Antimicrobial peptides, also known as lytic peptides or channel-forming peptides, are broad spectrum anti-bacterial agents. These peptides typically disrupt bacterial cell membranes, causing cell lysis and death. Over 100 antimicrobial peptides occur naturally. In addition, analogs have been synthesized de novo as described in Javadpour et al., J. Med. Chem. 39:3107-3113 (1996); and Blondelle and Houghten, Biochem. 31: 12688-12694 (1992), each of which is incorporated herein by reference. While some antimicrobial peptides such as melittin are not selective and damage normal mammalian cells at the minimum bactericidal concentration, others are selective for bacterial cells. For example, the naturally occurring magainins and cecropins exhibit substantial bactericidal activity at concentrations that are not lethal to normal mammalian cells.

Antimicrobial peptides frequently contain cationic amino acids, which are attracted to the head groups of anionic phospholipids, leading to the preferential disruption of negatively charged membranes. Once electrostatically bound, the amphipathic helices can distort the lipid matrix, resulting in loss of membrane barrier function (Epand, The Amphipathic Helix CRC Press: Boca Raton (1993); Lugtenberg and van Alphen, Biochim. Biophys. Acta 737:51-115 (1983), each of which is incorporated herein by reference). Prokaryotic cytoplasmic membranes maintain large transmembrane potentials and have a high content of anionic phospholipids. In contrast, the outer leaflet of eukaryotic plasma membranes generally has low, or no, membrane potential and is almost exclusively composed of zwitterionic phospholipids. Thus, due to distinct membrane compositions, antimicrobial peptides can preferentially disrupt prokaryotic membranes as compared to eukaryotic membranes.

The present invention is directed to the surprising discovery that an antimicrobial peptide sequence can be linked to a tumor homing molecule to produce a homing pro-apoptotic conjugate that generally is non-toxic outside of eukaryotic cells but which promotes disruption of mitochondrial membranes and subsequent cell death when targeted and internalized by eukaryotic cells. Homing pro-apoptotic conjugates such as HPP-1, which contains the antimicrobial peptide _{D}(KLAKLAK)₂ linked to the cyclic tumor homing molecule CNGRC (SEQ ID NO: 8), can have selective toxicity against angiogenic endothelial cells in vivo and, thus, be useful as a new class of anti-cancer therapeutics.

Thus, the present invention provides a homing pro-apoptotic conjugate, which includes a tumor homing molecule that selectively homes to a selected mammalian cell type or tissue linked to an antimicrobial peptide, where the conjugate is selectively internalized by the mammalian cell type or tissue and exhibits high toxicity thereto, and where the antimicrobial peptide has low mammalian cell toxicity when not linked to the tumor homing molecule. For example, a homing pro-apoptotic conjugate of the invention can exhibit selective toxicity against angiogenic endothelial cells and can be useful, for example, in methods of inducing selective toxicity in vivo in a tumor having angiogenic vasculature.

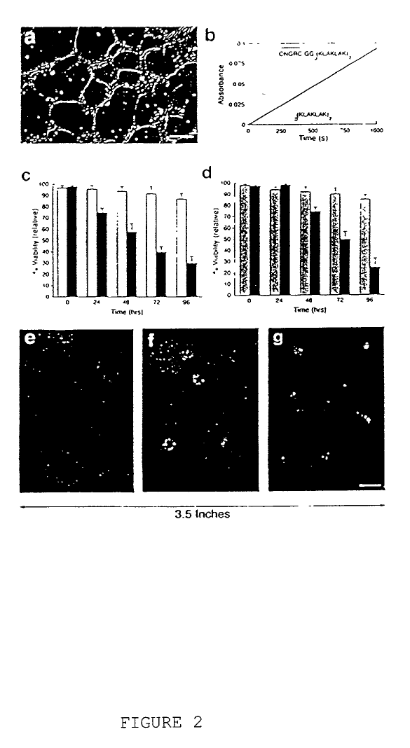

As disclosed herein, a synthetic antimicrobial peptide with selective toxicity against bacteria as compared to eukaryotic cells, _{D}(KLAKLAK)₂, induced marked mitochondrial swelling at a concentration of 10 μM (FIG. 2a), significantly less than the concentration required to kill eukaryotic cells, indicating that _{D}(KLAKLAK)₂ preferentially disrupts mitochondrial membranes as compared to eukaryotic membranes (see Example I). Moreover, _{D}(KLAKLAK)₂ activated mitochondria-dependent cell-free apoptosis as measured by characteristic caspase-3 processing (FIG. 2b) while a non-α-helix forming peptide DLSLARLATARLAI (SEQ ID NO: 204) did not. These results indicate that antimicrobial peptides such as _{D}(KLAKLAK)₂ can disrupt mitochondrial membranes, which, like bacterial membranes, have a high content of anionic phospholipids, reflecting the common ancestry of bacteria and mitochondria (Epand, supra, 1993; Lugtenberg and van Alphen, supra, 1983; Matsuzaki et al., Biochemistry 34:6521-6526 (1995); Hovius et al., FEBS Lett. 330:71-76 (1993); and Baltcheffsky and Baltcheffsky in Lee et al., Mitochondria and Microsomes Addison-Wesley: Reading, Mass. (1981), each of which is incorporated herein by reference).

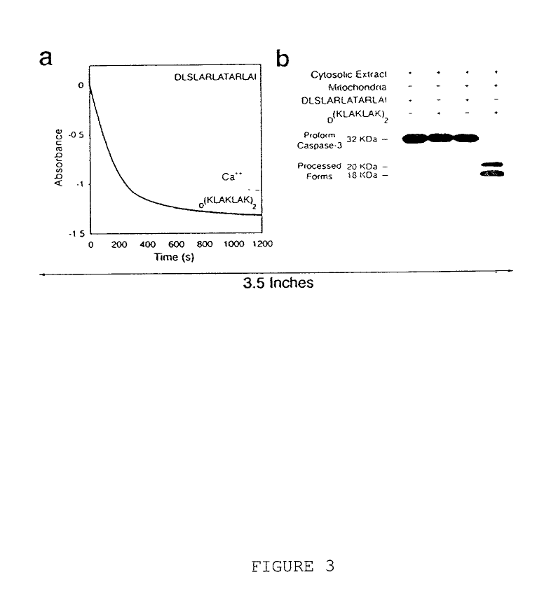

As further disclosed herein, the antimicrobial peptide _{D}(KLAKLAK)₂ was conjugated to the cyclic tumor homing peptide CNGRC (SEQ ID NO: 8) via a glycinylglycine bridge to produce the peptide CNGRC-GG-_{D}(KLAKLAK)₂, designated “HPP-1.” As disclosed herein, HPP-1 was tested in a tissue culture model of angiogenesis by assaying cord formation, which is a form of migration indicated by a change in endothelial cell morphology from the usual “cobblestones” to chains or cords of cells. Treatment of normal human dermal microvessel cells (DMECs) with 60 μM HPP-1 led to a decrease in percent viability with time under the conditions of proliferation (FIG. 3c) or cord formation (FIG. 3d), while treatment with untargeted _{D}(KLAKLAK)₂ peptide led to only a negligible loss in viability (see Example II). Furthermore, as shown in Table 1, the LC₅₀ for proliferating or migrating DMECs treated with HPP-1 was an order of magnitude lower than the LC₅₀ for angiostatic DMECs maintained in a monolayer at 100% confluency, demonstrating preferential killing by HPP-1 under angiogenic conditions. The results disclosed herein further demonstrate that the mitochondria of DMECs treated for 24 hours with _{D}(KLAKLAK)₂ remained morphologically normal, while those treated with CNGRC-GG-_{D}(KLAKLAK)₂ or ACDCRGDCFC-GG-_{D}(KLAKLAK)₂ displayed altered mitochondrial morphology before exhibiting the classical morphological indicators of apoptosis including nuclear condensation and fragmentation.

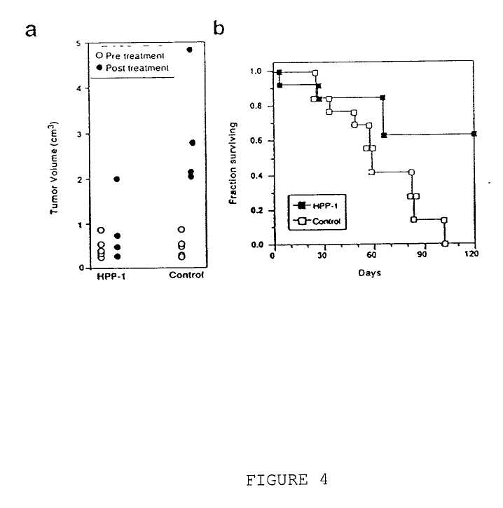

As shown in Example III, the HPP-1 peptide CNGRC-GG-_{D}(KLAKLAK)₂ also has activity in vivo. As disclosed in FIGS. 4a and b, nude mice bearing human MDA-MD-435 breast carcinoma xenografts were treated with HPP-1. Tumor volume was smaller on average by one order of magnitude, and survival longer in the HPP-1 treated animal groups as compared to control groups. Furthermore, some of the HPP-1 treated mice outlived control mice by several months, indicating that both primary tumor growth and metastasis were inhibited. Destruction of tumor architecture and widespread cell death was evident upon histopathological analysis of the tumors, with about 50% apoptotic cell death. HPP-1 also was effective against tumors derived from the human melanoma cell line C8161; and ACDCRGDCFC-GG-_{D}(KLAKLAK)₂ was effective against MDA-MD-435 breast carcinoma tumors. In sum, these results indicate that homing pro-apoptotic peptides based on tumor homing and antimicrobial peptide sequences can be non-toxic outside of eukaryotic cells but can promote disruption of mitochondrial membranes and subsequent cell death when internalized by the targeted eukaryotic target cells. Homing pro-apoptotic peptides such as HPP-1, which have selective toxicity against angiogenic endothelial cells, can be particularly valuable as anti-cancer therapeutics.

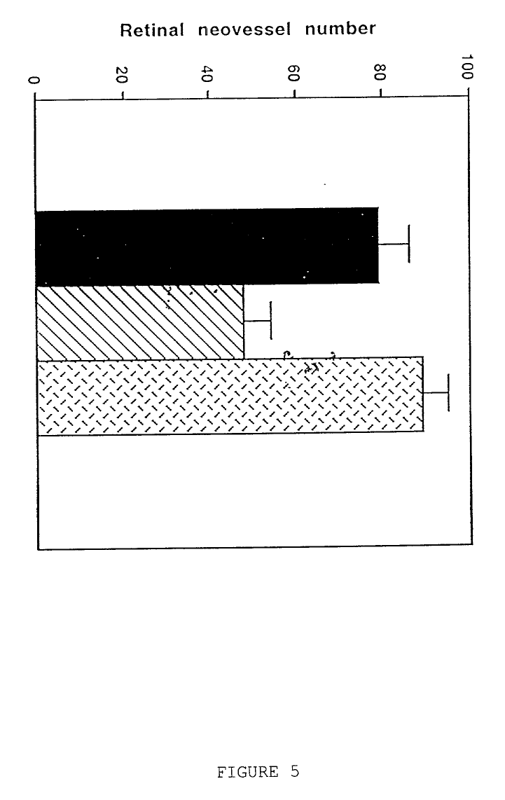

Further results disclosed herein show that retinal neovascularization can be selectively inhibited by a homing pro-apoptotic conjugate of the invention. In particular, the number of retinal neovessels in mice treated with the homing pro-apoptotic conjugate CDCRGDCFC-GG-_{D}(KLAKLAK)₂ was reduced to 30-40% of control levels (see FIG. 5). Thus, a homing pro-apoptotic conjugate of the invention can contain a tumor homing molecule, or can contain another homing molecule that selectively homes to a selected mammalian cell type or tissue.

A homing pro-apoptotic conjugate of the invention is characterized by being highly toxic to the mammalian cell type in which it is internalized. As used herein, the term “highly toxic” means that the conjugate is relatively effective in resulting in cell death of a selected cell type or tissue. One skilled in the art understands that toxicity can be analyzed using one of a variety of well known assays for cell viability. In general, the term highly toxic is used to refer to a conjugate in which the concentration for half maximal killing (LC₅₀) is less than about 100 μM, preferably less than about 50 μM. For example, as disclosed herein, the homing pro-apoptotic conjugate HPP-1 was characterized by LC₅₀s of 51, 34 and 42, respectively, for angiogenic proliferating and cord forming DMEM cells and for KS1767 cells. Moreover, the prolonged survival of tumor-bearing mice treating with a homing pro-apoptotic conjugate of the invention demonstrates that the selective toxicity can be reproduced in vivo.

As used herein, the term “antimicrobial peptide” means a naturally occurring or synthetic peptide having antimicrobial activity, which is the ability to kill or slow the growth of one or more microbes. An antimicrobial peptide can, for example, kill or slow the growth of one or more strains of bacteria including a Gram-positive or Gram-negative bacteria, or a fungi or protozoa. Thus, an antimicrobial peptide can have, for example, bacteriostatic or bacteriocidal activity against, for example, one or more strains of Escherichia coli, Pseudomonas aeruginosa or Staphylococcus aureus. While not wishing to be bound by the following, an antimicrobial peptide can have biological activity due to the ability to form ion channels through membrane bilayers as a consequence of self-aggregation.

An antimicrobial peptide is typically highly basic and can have a linear or cyclic structure. As discussed further below, an antimicrobial peptide can have an amphipathic α-helical structure (see U.S. Pat. No. 5,789,542; Javadpour et al., supra, 1996; Blondelle and Houghten, supra, 1992). An antimicrobial peptide also can be, for example, a β-strand/sheet-forming peptide as described in Mancheno et al., J. Peptide Res. 51:142-148 (1998).

An antimicrobial peptide can be a naturally occurring or synthetic peptide. Naturally occurring antimicrobial peptides have been isolated from biological sources such as bacteria, insects, amphibians and mammals and are thought to represent inducible defense proteins that can protect the host organism from bacterial infection. Naturally occurring antimicrobial peptides include the gramicidins, magainins, mellitins, defensins and cecropins (see, for example, Maloy and Kari, Biopolymers 37:105-122 (1995); Alvarez-Bravo et al., Biochem. J. 302:535-538 (1994); Bessalle et al., FEBS 274:151-155 (1990); and Blondelle and Houghten in Bristol (Ed.), Annual Reports in Medicinal Chemistry pages 159-168 Academic Press, San Diego, each of which is herein incorporated by reference). As discussed further below, an antimicrobial peptide also can be an analog of a natural peptide, especially one that retains or enhances amphipathicity.

An antimicrobial peptide incorporated within a homing pro-apoptotic conjugate of the invention has low mammalian cell toxicity when not linked to a tumor homing molecule. Mammalian cell toxicity readily can be assessed using routine assays. For example, mammalian cell toxicity can be assayed by lysis of human erythrocytes in vitro as described in Javadpour et al., supra, 1996. An antimicrobial peptide having “low mammalian cell toxicity” is not lytic to human erythrocytes or requires concentrations of greater than 100 μM for lytic activity, preferably concentrations greater than 200, 300, 500 or 1000 μM.

In a preferred embodiment, the invention also provides a homing pro-apoptotic conjugate in which the antimicrobial peptide portion promotes disruption of mitochondrial membranes when internalized by eukaryotic cells. In particular, such an antimicrobial peptide preferentially disrupts mitochondrial membranes as compared to eukaryotic membranes. Mitochondrial membranes, like bacterial membranes but in contrast to eukaryotic plasma membranes, have a high content of negatively charged phospholipids. An antimicrobial peptide can be assayed for activity in disrupting mitochondrial membranes using, for example, an assay for mitochondrial swelling (as described in Example I) or another assay well known in the art. As disclosed herein, for example, _{D}(KLAKLAK)₂ induced marked mitochondrial swelling at a concentration of 10 μM, significantly less than the concentration required to kill eukaryotic cells. An antimicrobial peptide that induces significant mitochondrial swelling at, for example, 50 μM, 40 μM, 30 μM, 20 μM, 10 μM, or less, is considered a peptide that promotes disruption of mitochondrial membranes.

The invention also provides a homing pro-apoptotic conjugate in which a tumor homing molecule is linked to an antimicrobial peptide having an amphipathic α-helical structure. In a homing pro-apoptotic conjugate of the invention, the antimicrobial peptide portion can have, for example, the sequence (KLAKLAK)₂ (SEQ ID NO: 200); (KLAKKLA)₂ (SEQ ID NO: 201) (KAAKKAA)₂ (SEQ ID NO: 202); or (KLGKKLG)₃ (SEQ ID NO: 203), in particular the sequence _{D}(KLAKLAK)₂. A homing pro-apoptotic conjugate of the invention can have, for example, the sequence CNGRC-GG-_{D}(KLAKLAK)₂ or ACDCRGDCFC-GG-_{D}(KLAKLAK)₂.

Antimicrobial peptides generally have random coil conformations in dilute aqueous solutions, yet high levels of helicity can be induced by helix-promoting solvents and amphipathic media such as micelles, synthetic bilayers or cell membranes. α-Helical structures are well known in the art, with an ideal α-helix characterized by having 3.6 residues per turn and a translation of 1.5 Å per residue (5.4 Å per turn; see Creighton, Proteins: Structures and Molecular Properties W.H Freeman, New York (1984)). In an amphipathic α-helical structure, polar and non-polar amino acid residues are aligned into an amphipathic helix, which is an α-helix in which the hydrophobic amino acid residues are predominantly on one face, with hydrophilic residues predominantly on the opposite face when the peptide is viewed along the helical axis.

Antimicrobial peptides of widely varying sequence have been isolated, sharing an amphipathic α-helical structure as a common feature (Saberwal et al., Biochim. Biophys. Acta 1197:109-131 (1994)). Analogs of native peptides with amino acid substitutions predicted to enhance amphipathicity and helicity typically have increased antimicrobial activity. In general, analogs with increased antimicrobial activity also have increased cytotoxicity against mammalian cells (Maloy et al., Biopolymers 37:105-122 (1995)).

As used herein in reference to an antimicrobial peptide, the term “amphipathic α-helical structure” means an α-helix with a hydrophilic face containing several polar residues at physiological pH and a hydrophobic face containing nonpolar residues. A polar residue can be, for example, a lysine or arginine residue, while a nonpolar residue can be, for example, a leucine or alanine residue. An antimicrobial peptide having an amphipathic α-helical structure generally has an equivalent number of polar and nonpolar residues within the amphipathic domain and a sufficient number of basic residues to give the peptide an overall positive charge at neutral pH (Saberwal et al., Biochim. Biophys. Acta 1197:109-131 (1994), which is incorporated by reference herein). One skilled in the art understands that helix-promoting amino acids such as leucine and alanine can be advantageously included in an antimicrobial peptide of the invention (see, for example, Creighton, supra, 1984).

A variety of antimicrobial peptides having an amphipathic α-helical structure are well known in the art. Such peptides include synthetic, minimalist peptides based on a heptad building block scheme in which repetitive heptads are composed of repetitive trimers with an additional residue. Such synthetic antimicrobial peptides include, for example, peptides of the general formula [(X₁X₂X₂)(X₁X₂X₂)X₁]ₙ (SEQ ID NO: 205) or [(X₁X₂X₂)X₁(X₁X₂X₂)]ₙ (SEQ ID NO: 206), where X₁ is a polar residue, X₂ is a nonpolar residue; and n is 2 or 3 (see Javadpour et al., supra, 1996. (KLAKLAK)₂ (SEQ ID NO: 200); (KLAKKLA)₂ (SEQ ID NO: 201); (KAAKKAA)₂ (SEQ ID NO: 202); and (KLGKKLG)₃ (SEQ ID NO: 203) are examples of synthetic antimicrobial peptides having an amphipathic α-helical structure. Similar synthetic, antimicrobial peptides having an amphipathic α-helical structure also are known in the art, for example, as described in U.S. Pat. No. 5,789,542 to McLaughlin and Becker.

Helicity readily can be determined by one skilled in the art, for example, using circular dichroism spectroscopy. Percent α-helicity can be determined, for example, after measuring molar ellipticity at 222 nm as described in Javadpour et al., supra, 1996 (see, also, McLean et al., Biochemistry 30:31-37 (1991), which is incorporated by reference herein). An amphipathic α-helical antimicrobial peptide of the invention can have, for example, at least about 20% helicity when assayed in amphipathic media such as 25 mM SDS. One skilled in the art understands that such an antimicrobial peptide having an amphipathic α-helical structure can have, for example, at least about 25%, 30%, 35% or 40% helicity when assayed in 25 mM SDS. An antimicrobial peptide having an α-helical structure can have, for example, from 25% to 90% helicity; 25% to 60% helicity; 25% to 50% helicity; 25% to 40% helicity; 30% to 90% helicity; 30% to 60% helicity; 30% to 50% helicity; 40% to 90% helicity or 40% to 60% helicity when in assayed in 25 mM SDS. Amphipathicity can readily be determined, for example, using a helical wheel representation of the peptide (see, for example, Blondelle and Houghten, supra, 1994).

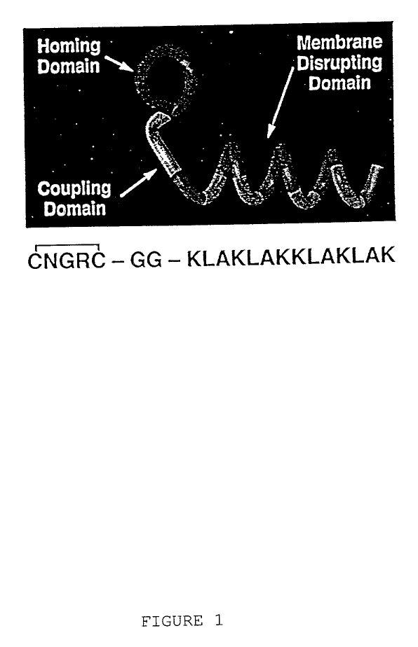

The structure of an exemplary homing pro-apoptotic conjugate of the invention, CNGRC-GG-_{D}(KLAKLAK)₂ is illustrated in FIG. 1. As can be seen in FIG. 1, the homing domain, CNGRC (SEQ ID NO: 8) is a disulfide-bonded cyclic structure and is coupled to a membrane disrupting domain, _{D}(KLAKLAKKLAKLAK) via a glycinylglycine bridge. The D-amino acids in the membrane disrupting, antimicrobial portion of the conjugate can be useful in imparting increased stability upon the conjugate in vivo. Furthermore, the membrane disrupting _{D}(KLAKLAKKLAKLAK) portion forms an amphipathic helix. In particular, the lysine residues are aligned on one face of the helix (shown as dark shaded region of helix), while the non-polar leucine and alanine residues are aligned on the opposite (light-shaded) face of the helix.

A homing pro-apoptotic conjugate of the invention can be a chimeric peptide in which the tumor homing molecule is a tumor homing peptide. A homing pro-apoptotic chimeric peptide of the invention can have a variety of lengths, from about 18 amino acids to about fifty amino acids or more. A chimeric peptide of the invention can have, for example, from about 20 to about fifty amino acids, preferably from 20 to 40 amino acids, more preferably from 20 to 30 amino acids. Such a chimeric peptide can have, for example, an upper length of 40, 35, 30, 27, 25 or 21 amino acids. A chimeric peptide of the invention can be linear or cyclic. In a preferred embodiment, a homing pro-apoptotic chimeric peptide of the invention includes a cyclic tumor homing peptide portion.

A homing pro-apoptotic chimeric peptide of the invention also can be a peptidomimetic. As used herein, the term “peptidomimetic” is used broadly to mean a peptide-like molecule that has substantially the activity of the corresponding peptide. Peptidomimetics include chemically modified peptides, peptide-like molecules containing non-naturally occurring amino acids, peptoids and the like, have the selective homing activity and the high toxicity of the peptide from which the peptidomimetic is derived (see, for example, “Burger's Medicinal Chemistry and Drug Discovery” 5th ed., vols. 1 to 3 (ed. M. E. Wolff; Wiley Interscience 1995), which is incorporated herein by reference). For example, D amino acids can be advantageously included in the antimicrobial peptide portion of a chimeric peptide of the invention (see Examples I and II). Peptidomimetics provide various advantages over a peptide, including increased stability during passage through the digestive tract and, therefore, can be advantageously used as oral therapeutics.

In a homing pro-apoptotic conjugate of the invention, a “coupling domain” can be used to link a tumor homing peptide and an antimicrobial peptide and can, for example, impart flexibility to the conjugate as a whole. A coupling domain can be, for example, a glycinylglycine linker, alaninylalanine linker or other linker incorporating glycine, alanine or other amino acids. The use of a glycinylglycine coupling domain is described in Example II.

The vasculature within a tumor generally undergoes active angiogenesis, resulting in the continual formation of new blood vessels to support the growing tumor. Such angiogenic blood vessels are distinguishable from mature vasculature in that angiogenic vasculature expresses unique endothelial cell surface markers, including the αᵥβ₃ integrin (Brooks, Cell 79:1157-1164 (1994); WO 95/14714, Int. Filing Date Nov. 22, 1994) and receptors for angiogenic growth factors (Mustonen and Alitalo, J. Cell Biol. 129:895-898 (1995); Lappi, Semin. Cancer Biol. 6:279-288 (1995)). Moreover, tumor vasculature is histologically distinguishable from other blood vessels in that tumor vasculature is fenestrated (Folkman, Nature Med. 1:27-31 (1995); Rak et al., Anticancer Drugs 6:3-18 (1995)). Thus, the unique characteristics of tumor vasculature make it a particularly attractive target for anti-cancer therapeutics.

As disclosed herein, tumor homing molecules can bind to the endothelial lining of small blood vessels of tumors. The vasculature within tumors is distinct, presumably due to the continual neovascularization, resulting in the formation of new blood vessels required for tumor growth. The distinct properties of the angiogenic neovasculature within tumors are reflected in the presence of specific markers in endothelial cells and pericytes (Folkman, Nature Biotechnol. 15:510 (1997); Risau, FASEB J. 9:926-933 (1995); Brooks et al., supra, 1994); these markers likely are being targeted by the disclosed tumor homing molecules.

The ability of a tumor homing molecule to target the blood vessels in a tumor provides substantial advantages over methods of systemic treatment or methods that directly target the tumor cells. For example, tumor cells depend on a vascular supply for survival and the endothelial lining of blood vessels is readily accessible to a circulating probe. Conversely, in order to reach solid tumor cells, a therapeutic agent must overcome potentially long diffusion distances, closely packed tumor cells, and a dense fibrous stroma with a high interstitial pressure that impedes extravasation (Burrows and Thorpe, Pharmacol. Ther. 64:155-174 (1994)).

In addition, where the tumor vasculature is targeted, the killing of all target cells may not be required, since partial denudation of the endothelium can lead to the formation of an occlusive thrombus halting the blood flow through the entirety of the affected tumor vessel (Burrows and Thorpe, supra, 1994). Furthermore, unlike direct tumor targeting, there is an intrinsic amplification mechanism in tumor vasculature targeting. A single capillary loop can supply nutrients to up to 100 tumor cells, each of which is critically dependent on the blood supply (Denekamp, Cancer Metast. Rev. 9:267-282 (1990); Folkman, supra, 1997).

As set forth above and exemplified herein, a tumor homing molecule that is selective for the angiogenic endothelial cells of tumor vasculature can be particularly useful for directing a pro-apoptotic antimicrobial peptide to tumor vasculature, while reducing the likelihood that the pro-apoptotic antimicrobial peptide will have a toxic effect on normal, healthy organs or tissues. Thus, in one embodiment, the invention provides a homing pro-apoptotic conjugate, which includes a tumor homing molecule that selectively homes to angiogenic endothelial cells linked to an antimicrobial peptide, where the conjugate is selectively internalized by angiogenic endothelial cells and exhibits high toxicity thereto, and where the antimicrobial peptide has low mammalian cell toxicity when not linked to the tumor homing molecule.

As used herein, the term “selective toxicity” means enhanced cell death in a selected cell type or tissue as compared to a control cell type or tissue. In general, selective toxicity is characterized by at least a two-fold greater extent of cell death in the selected cell type or tissue, such as angiogenic endothelial cells, as compared to a control cell type or tissue, for example, angiostatic endothelial cells. Thus, as used herein, the term selective toxicity encompasses specific toxicity, whereby cell death occurs essentially only the selected cell type or tissue, as well as toxicity occurring in a limited number of cell types or tissues in addition to the selected cell type or tissue. One skilled in the art further understands that the term selective toxicity refers to cell death effected by all mechanisms including apoptotic and necrotic cell death. Thus, a homing pro-apoptotic conjugate of the invention that exhibits selective toxicity for angiogenic endothelial cells effects enhanced cell death of the angiogenic endothelial cells as compared to angiostatic endothelial cells or surrounding cells of other types.

As disclosed herein, identified tumor homing molecules are useful for targeting a desired antimicrobial peptide, which is linked to the homing molecule, to a selected cell type such as angiogenic endothelial cells. After being internalized by the angiogenic endothelial cells in tumor vasculature, the antimicrobial peptide is toxic to the endothelial cells, thereby restricting the blood supply to the tumor and inhibiting tumor growth.

A tumor homing molecule useful in the homing pro-apoptotic conjugates of the invention can be a peptide containing, for example, an NGR motif, such as CNGRC (SEQ ID NO: 8); NGRAHA (SEQ ID NO: 6) or CNGRCVSGCAGRC (SEQ ID NO: 3). A tumor homing molecule useful in the invention also can contain an RGD motif and can be, for example, CDCRGDCFC (SEQ ID NO: 1), or can contain a GSL motif, such as the peptide CGSLVRC (SEQ ID NO: 5). Additional tumor homing molecules can be identified by screening a library of molecules by in vivo panning as set forth in further detail below (see, also, Examples IV to VIII; U.S. Pat. No. 5,622,699, issued Apr. 22, 1997; and Pasqualini and Ruoslahti, Nature 380:364-366 (1996), each of which is incorporated herein by reference).

The term “tumor homing molecule,” as used herein, means an organic chemical such as a drug; a nucleic acid molecule; a peptide or peptidomimetic or protein that selectively homes in vivo to a selected cell type or tissue. By “selectively homes” is meant that, in vivo, the tumor homing molecule binds preferentially to a selected cell type or tissue as compared to a control cell type, tissue or organ and generally is characterized by at least a two-fold greater localization at the selected cell type or tissue compared to a control cell type or tissue. A tumor homing molecule useful in the invention can be, for example, a molecule that binds preferentially to the endothelial cells of angiogenic vasculature as compared to other cell types or angiostatic vasculature.

Tumor homing molecules were identified using in vivo panning as follows. By panning in vivo against a breast carcinoma, a melanoma and a Kaposi's sarcoma, phage expressing various peptides that selectively homed to tumors were identified (see Tables 2, 3 and 4, respectively). Due to the large size of the phage (900-1000 nm) and the short time the phage were allowed to circulate (3 to 5 min), it is unlikely that a substantial number of phage would have exited the circulatory system, particularly in the brain and kidney. Tissue staining studies indicated that the tumor homing molecules that were identified primarily homed to and bound endothelial cell surface markers, which likely are expressed in an organ-specific manner. These results indicate that in vivo panning can be used to identify and analyze endothelial cell specificities. Such an analysis is not possible using endothelial cells in culture because the cultured cells tend to lose their tissue-specific differences (Pauli and Lee, Lab. Invest. 58:379-387 (1988)).

Although the conditions under which the in vivo pannings were performed identified tumor homing peptides that generally bind to endothelial cell markers, the specific presence of phage expressing tumor homing peptides also was observed in tumor parenchyma, particularly at later times after administration of the peptides (Example VII). These results demonstrate that phage expressing peptides can pass through the blood vessels in the tumor, possibly due to the fenestrated nature of the blood vessels, and indicate that the in vivo panning method can be useful for identifying target molecules expressed by tumor cells, as well as target molecules expressed by endothelial cells.

Phage peptide display libraries were constructed essentially as described in Smith and Scott (supra, 1993; see, also, Koivunen et al., Biotechnology 13:265-270 (1995); Koivunen et al., Meth. Enzymol. 245:346-369 (1994b), each of which is incorporated herein by reference). Oligonucleotides encoding peptides having substantially random amino acid sequences were synthesized based on an “NNK” codon, wherein “N” is A, T, C or G and “K” is G or T. “NNK” encodes 32 triplets, which encode the twenty amino acids and an amber STOP codon (Scott and Smith, supra, 1990). In some libraries, at least one codon encoding cysteine also was included in each oligonucleotide so that cyclic peptides could be formed through disulfide linkages (Example IV). The oligonucleotides were inserted in frame with the sequence encoding the gene III protein (gIII) in the vector fuse 5 such that a peptide-gIII fusion protein was expressed. Following expression, the fusion protein was expressed on the surface of the phage containing the vector (Koivunen et al., supra, 1994b; Smith and Scott, supra, 1993).

Following in vivo panning, the phage isolated based on their ability to selectively home to human breast carcinoma, mouse melanoma or human Kaposi's sarcoma tumors displayed only a few different peptide sequences (see Tables 2, 3 and 4, respectively). One of the screenings revealed peptide sequences that contained the arginine-glycine-aspartic acid (RGD) integrin recognition sequence (Ruoslahti, Ann. Rev. Cell Devel. Biol. 12:697 (1996)) in the context of a peptide previously demonstrated to bind selectively to αᵥ-containing integrins (Koivunen et al., supra, 1995; WO 95/14714). The sequences of most of the remaining tumor homing peptides did not reveal any significant similarities with known ligands for endothelial cell receptors. However, one of the tumor homing peptides contained the asparagine-glycine-arginine (NGR) motif, which is a weak integrin binding motif similar to the motifs present in integrin-binding peptides (Ruoslahti et al., U.S. Pat. No. 5,536,814, issued Jul. 16, 1996, which is incorporated herein by reference; see, also, Koivunen et al., supra, 1994a). Other screenings have revealed numerous NGR-containing peptides (see Table 2). Despite the weak integrin binding ability of NGR peptides, an integrin receptor may not be the target molecule recognized by the NGR tumor homing peptides exemplified herein. As used herein, the term “integrin” means a heterodimeric cell surface adhesion receptor.

The peptides expressed by the phage that homed to the breast tumor included the peptides CGRECPRLCQSSC (SEQ ID NO: 2) and CNGRCVSGCAGRC (SEQ ID NO: 3; see Table 2; Example V). Similarly, tumor homing peptides, including the peptides CDCRGDCFC (SEQ ID NO: 1) and CGSLVRC (SEQ ID NO: 5), were identified from two other phage libraries administered to breast tumor bearing mice (Table 2). Some of these motifs, as well as novel ones, also were isolated by screening against mouse melanoma and human Kaposi's sarcoma (see Tables 3 and 4). These results demonstrated that tumor homing molecules can be identified using in vivo panning.

Three main tumor homing motifs that were identified can be particularly useful in the homing pro-apoptotic conjugates of the invention. Homing pro-apoptotic conjugates in which the tumor homing molecule portion contains an NGR motif, RGD motif or GSL motif, can be used to target a linked antimicrobial peptide to the endothelial cells of angiogenic vasculature.

In one embodiment, the invention provides a homing pro-apoptotic conjugate, which includes a tumor homing peptide containing the sequence NGR linked to an antimicrobial peptide. In such a homing pro-apoptotic conjugate of the invention, the tumor homing peptide can be, for example, CNGRC (SEQ ID NO: 8); NGRAHA (SEQ ID NO: 6) or CNGRCVSGCAGRC (SEQ ID NO: 3). In a preferred embodiment, the homing pro-apoptotic conjugate includes the sequence CNGRC-GG-_{D}(KLAKLAK)₂.

In another embodiment, the invention provides a homing pro-apoptotic conjugate, which includes a tumor homing peptide containing the sequence RGD linked to an antimicrobial peptide. In such a homing pro-apoptotic conjugate, the tumor homing peptide can be, for example, CDCRGDCFC (SEQ ID NO: 1). In a preferred embodiment, the homing pro-apoptotic conjugate includes the sequence ACDCRGDCFC-GG-_{D}(KLAKLAK)₂.

The invention additionally provides a homing pro-apoptotic conjugate, which includes a tumor homing peptide containing the sequence GSL linked to an antimicrobial peptide. In such a homing pro-apoptotic conjugate, the tumor homing peptide can be, for example, CGSLVRC (SEQ ID NO: 5).

As discussed above, one motif contained the sequence RGD (Ruoslahti, supra, 1996) embedded in the peptide structure, CDCRGDCFC (SEQ ID NO: 1), which is known to bind selectively to αᵥ integrins (Koivunen et al., supra, 1995; WO 95/14714). Since the αᵥβ₃ and αᵥβ₅ integrins are markers of angiogenic vessels (Brooks et al., supra, 1994; Friedlander et al., Science 270:1500 (1995)), a phage expressing the peptide CDCRGDCFC (SEQ ID NO: 1) was examined for tumor targeting and, as disclosed herein, homed to tumors in a highly selective manner (see Example VI). Furthermore, homing by the CDCRGDCFC (SEQ ID NO: 1) phage was inhibited by coadministration of the free CDCRGDCFC (SEQ ID NO: 1) peptide.

Another breast tumor homing peptide had the sequence CNGRCVSGCAGRC (SEQ ID NO: 3), which contains the NGR motif previously shown to have weak integrin binding activity (Koivunen et al., J. Biol. Chem. 268:20205-20210 (1993); Koivunen et al., supra, 1994a; WO 95/14714). Since an NGR containing peptide was identified, two additional peptides, the linear peptide, NGRAHA (SEQ ID NO: 6), and the cyclic peptide, CVLNGRMEC (SEQ ID NO: 7), each of which contains the NGR motif, were examined for tumor homing. Like the phage expressing CNGRCVSGCAGRC (SEQ ID NO: 3), phage expressing NGRAHA (SEQ ID NO: 6) or CVLNGRMEC (SEQ ID NO: 7) homed to the tumors. Furthermore, tumor homing was not dependent on the tumor type or on species, as the phage accumulated selectively in human breast carcinoma, as well as in the tumors of mice bearing a mouse melanoma and mice bearing a human Kaposi's sarcoma xenograft.

The various peptides, including RGD- and NGR-containing peptides, generally were bound to the tumor blood vessels. The minimal cyclic NGR peptide, CNGRC (SEQ ID NO: 8), was synthesized based on the CNGRCVSGCAGRC (SEQ ID NO: 3) sequence. When the CNGRC (SEQ ID NO: 8) peptide was co-injected with phage expressing either CNGRCVSGCAGRC (SEQ ID NO: 3), NGRAHA (SEQ ID NO: 6) or CVLNGRMEC (SEQ ID NO: 7), accumulation of the phage in the breast carcinoma xenografts was inhibited. However, the CNGRC (SEQ ID NO: 8) peptide did not inhibit the homing of phage expressing the CDCRGDCFC (SEQ ID NO: 1) peptide, even when administered in amounts up to ten times higher than those that inhibited the homing of the NGR phage. In comparison, the CDCRGDCFC (SEQ ID NO: 1) peptide partially inhibited the homing of the NGR phage, although the amount needed was 5 to 10 fold higher than that of the CNGRC peptide (SEQ ID NO: 8). These results indicate that NGR peptides and RGD peptides bind to different receptor sites in tumor vasculature.

A third motif, GSL (glycine-serine-leucine), also was identified following in vivo panning in mice bearing breast carcinoma, malignant melanoma or Kaposi's sarcoma. Homing of phage expressing the GSL peptide, CGSLVRC (SEQ ID NO: 5), was inhibited by coadministration of the free CGSLVRC (SEQ ID NO: 5) peptide. Like the RGD and NGR peptides, phage expressing GSL peptides also bound to blood vessels of tumors. In view of the identification of the conserved RGD, NGR and GSL motifs present in tumor homing peptides, as disclosed herein, it will be recognized that peptides containing such motifs can be useful as tumor homing peptides and, in particular, for forming homing pro-apoptotic conjugates that can selectively deliver an antimicrobial peptide to a tumor.

Various peptide libraries containing up to 13 amino acids were constructed, and the NGR peptide, CNGRCVSGCAGRC (SEQ ID NO: 3), was obtained as a result of in vivo panning against a breast tumor. This NGR peptide, which was obtained by screening a random peptide library, was a tumor homing peptide. In addition, when a peptide library was constructed based on the formula CXXXNGRXX (SEQ ID NO: 13) or CXXCNGRCX (SEQ ID NO: 14), each of which is biased toward NGR sequences, and used for in vivo panning against a breast tumor, numerous NGR peptides were obtained (see Table 2).

These results indicate that a tumor homing molecule of the invention can comprise the amino acid sequence RGD or NGR or GSL. Such a tumor homing molecule can be a peptide as small as five amino acids, such as CNGRC (SEQ ID NO: 8). Such tumor homing peptides also can be not only at least 13 amino acids in length, which is the largest peptide exemplified herein, but can be up to 20 amino acids, or 30 amino acids, or 50 to 100 amino acids in length, as desired. A tumor homing peptide of the invention conveniently is produced by chemical synthesis.

Immunohistochemical analysis was performed by comparing tissue staining for phage allowed to circulate for about four minutes, followed by perfusion through the heart of the mice, or with tissues analyzed 24 hours after phage injection. At 24 hours following administration, essentially no phage remain in the circulation and, therefore, perfusion is not required (Pasqualini et al., supra, 1997). Strong phage staining was observed in tumor vasculature, but not in normal endothelium, in samples examined four minutes after administration of the CNGRCVSGCAGRC (SEQ ID NO: 3) phage (Example VII). In comparison, staining of the tumor was strong at 24 hours and appeared to have spread outside the blood vessels into the tumor parenchyma. The NGRAHA (SEQ ID NO: 6) and CVLNGRMEC (SEQ ID NO: 7) phage showed similar staining patterns (Example VII). In contrast, the control organs and tissues showed little or no immunostaining, confirming the specificity of the NGR motifs for tumor vessels. Spleen and liver, however, captured phage, as expected, since uptake by the reticuloendothelial system is a general property of phage particles, independent of the presence of peptide expression by the phage (Pasqualini et al., supra, 1997).

Immunostaining also was observed following administration of phage expressing the GSL motif containing peptide, CLSGSLSC (SEQ ID NO: 4), and, like that of the NGR peptides, was localized to the blood vessels, in this case, within a melanoma tumor (see below; see, also, Examples VII and VIII). Similarly, immunostaining following administration of phage expressing the RGD motif containing peptide, CDCRGDCFC (SEQ ID NO: 1), to breast tumor bearing mice was localized to the blood vessels in the tumor, but was not observed in brain, kidney or various other nontumor tissues (see Examples VI and VII; see, also, Pasqualini et al., supra, 1997). These results demonstrate that the various tumor homing peptides generally home to tumor vasculature.

The general applicability of the in vivo panning method for identifying tumor homing molecules was examined by injecting mice bearing a syngeneic melanoma with phage expressing a diverse population of peptides (Example VIII). The B16 mouse melanoma model was selected for these studies because the tumors that form are highly vascularized and because the biology of this tumor line has been thoroughly characterized (see Miner et al., Cancer Res. 42:4631-4638 (1982)). Furthermore, because the B16 melanoma cells are of mouse origin, species differences between the host and the tumor cell donor will not affect, for example, the distribution of phage into the tumor as compared to into normal organs. As disclosed herein, in vivo panning against B16 melanoma cells revealed tumor homing peptides, including, for example, the GSL moiety containing peptide CLSGSLSC (SEQ ID NO: 4; see, also, Table 3) and immunohistochemical staining of the tumor and other organs using an anti-phage antibody demonstrated that the CLSGSLSC (SEQ ID NO: 4) expressing phage resulted in immunostaining in the melanoma, but essentially no staining in skin, kidney or other control organs (Example VIII). The staining pattern generally followed the blood vessels within the melanoma, but was not strictly confined to the blood vessels.

Although in vivo panning was performed in mice, tumor homing molecules such as peptides comprising an NGR, RGD or GSL motif also likely can target human vasculature. The NGR phage binds to blood vessels in the transplanted human breast tumor, but not to blood vessels in normal tissues, indicating that this motif can be particularly useful for tumor targeting in patients. The CDCRGDCFC (SEQ ID NO: 1) peptide binds to human αᵥ-integrins (Koivunen et al., supra, 1995), which are selectively expressed in tumor blood vessels of human patients (Max et al., Int. J. Cancer 71:320 (1997); Max et al., Int. J. Cancer 72:706 (1997)). Use of a homing pro-apoptotic conjugate in which CDCRGDCFC (SEQ ID NO: 1) is linked to an antimicrobial peptide provides the additional advantage that the antimicrobial peptide can be targeted to tumor cells, themselves, because breast carcinoma cells, for example, can express the αᵥβ₃ integrin (Pasqualini et al., supra, 1997). In fact, many human tumors express this integrin, which may be involved in the progression of certain tumors such as malignant melanomas (Albelda et al., Cancer Res. 50:6757-6764 (1990); Danen et al., Int. J. Cancer 61:491-496 (1995); Felding-Habermann et al., J. Clin. Invest. 89:2018-2022 (1992); Sanders et al., Cold Spring Harb. Symp. Ouant. Biol. 58:233-240 (1992); Mitjans et al., J. Cell. Sci. 108:3067-3078 (1995)). Unlike the CDCRGDCFC (SEQ ID NO: 1) peptide, the NGR peptides do not appear to bind to MDA-MD-435 breast carcinoma cells. However, NGR peptides were able to deliver a therapeutically effective amount of doxorubicin to breast tumors, indicating that, even where a tumor homing molecule homes only to tumor vasculature, i.e., not directly to the tumor cells, such vasculature targeting in sufficient to confer the effect of the moiety linked to the molecule.

Since the αᵥβ₃ integrin is expressed by endothelial cells in angiogenic vasculature, experiments were performed to determine whether tumor vasculature that is undergoing angiogenesis can be targeted in vivo using methods as disclosed herein. Phage expressing the peptide, CDCRGDCFC (SEQ ID NO: 1; see, Koivunen et al., supra, 1995), which is known to bind to the αᵥβ₃ integrin, were injected into mice bearing tumors formed from human breast carcinoma cells, mouse melanoma cells or human Kaposi's sarcoma cells (see Example VII). The CDCRGDCFC (SEQ ID NO: 1) phage selectively homed to each of the tumors, whereas such homing did not occur with control phage. For example, in mice bearing tumors formed by implantation of human breast carcinoma cells, a twenty- to eighty-fold greater number of the CDCRGDCFC (SEQ ID NO: 1) phage, as compared to unselected control phage, accumulated in the tumor.

Tissue staining for the phage showed accumulation of the CDCRGDCFC (SEQ ID NO: 1) phage in the blood vessels within the tumor, whereas no staining was observed in brain, kidney or other control organs. Specificity of tumor homing by the CDCRGDCFC (SEQ ID NO: 1) phage was demonstrated by competition experiments, in which coinjection of the free CDCRGDCFC (SEQ ID NO: 1) peptide greatly reduced tumor homing of the RGD phage, whereas coinjection of a non-RGD-containing control peptide had no effect on homing of the RGD phage (see Example VI). These results demonstrate that the αᵥβ₃ target molecule is expressed on the luminal surface of endothelial cells in a tumor and that a peptide that binds to an αᵥ-containing integrin can bind selectively to this integrin and, therefore, to vasculature undergoing angiogenesis.

The results of these studies indicate that tumor homing molecules can be identified by in vivo panning and that, in some cases, a tumor homing molecule can home to vascular tissue in the tumor as well as to tumor parenchyma, probably due to the fenestrated nature of the blood vessels permitting ready exit of the phage from the circulatory system. Due to the ability of such tumor homing molecules to home to tumors, the molecules are useful for targeting a linked antimicrobial peptide to tumors. Thus, the invention provides conjugates comprising a tumor homing molecule linked to a moiety, such conjugates being useful for targeting the moiety to tumor cells.

The ability of a molecule that homes to a particular tumor to selectively home to another tumor of the same or a similar histologic type can be determined using, for example, human tumors grown in nude mice or mouse tumors grown in syngeneic mice for these experiments. For example, various human breast cancer cell lines, including MDA-MB-435 breast carcinoma (Price et al., Cancer Res. 50:717-721 (1990)), SKBR-1-II and SK-BR-3 (Fogh et al., J. Natl. Cancer Inst. 59:221-226 (1975)), and mouse mammary tumor lines, including EMT6 (Rosen et al., Int. J. Cancer 57:706-714 (1994)) and C3-L5 (Lala and Parhar, Int. J. Cancer 54:677-684 (1993)), are readily available and commonly used as models for human breast cancer. Using such breast tumor models, for example, information relating to the specificity of an identified breast tumor homing molecule for diverse breast tumors can be obtained and molecules that home to a broad range of different breast tumors or provide the most favorable specificity profiles can be identified. In addition, such analyses can yield new information, for example, about tumor stroma, since stromal cell gene expression, like that of endothelial cells, can be modified by the tumor in ways that cannot be reproduced in vitro.

Selective homing of a molecule such as a peptide or protein to a tumor can be due to specific recognition by the peptide of a particular cell target molecule such as a cell surface receptor present on a cell in the tumor. Selectivity of homing is dependent on the particular target molecule being expressed on only one or a few different cell types, such that the molecule homes primarily to the tumor. As discussed above, the identified tumor homing peptides, at least in part, can recognize endothelial cell surface markers in the blood vessels present in the tumors. However, most cell types, particularly cell types that are unique to an organ or tissue, can express unique target molecules. Thus, in vivo panning can be used to identify molecules that selectively home to a particular type of tumor cell such as a breast cancer cell; specific homing can be demonstrated by performing the appropriate competition experiments.

As used herein, the term “tumor” means a mass of cells that are characterized, at least in part, by containing angiogenic vasculature. The term “tumor” is used broadly to include the tumor parenchymal cells as well as the supporting stroma, including the angiogenic blood vessels that infiltrate the tumor parenchymal cell mass. Although a tumor generally is a malignant tumor, i.e., a “cancer,” a tumor also can be nonmalignant, provided that neovascularization is associated with the tumor. The term “normal” or “nontumor” tissue is used to refer to tissue that is not a “tumor.” As disclosed herein, a tumor homing molecule can be identified based on its ability to home a tumor, but not to a corresponding nontumor tissue.

As used herein, the term “corresponding,” when used in reference to tumors or tissues or both, means that two or more tumors, or two or more tissues, or a tumor and a tissue are of the same histologic type. The skilled artisan will recognize that the histologic type of a tissue is a function of the cells comprising the tissue. Thus, the artisan will recognize, for example, that a nontumor tissue corresponding to a breast tumor is normal breast tissue, whereas a nontumor tissue corresponding to a melanoma is skin, which contains melanocytes. Furthermore, for purposes of the invention, it is recognized that a tumor homing molecule can bind specifically to a target molecule expressed by the vasculature in a tumor, which generally contains blood vessels undergoing neovascularization, in which case a tissue corresponding to the tumor would comprise nontumor tissue containing blood vessels that are not undergoing active angiogenesis.

A tumor homing molecule useful in the invention can be identified by screening a library of molecules by in vivo panning as disclosed herein and set forth in U.S. Pat. No. 5,622,699, issued Apr. 22, 1997; and Pasqualini and Ruoslahti, Nature 380:364-366 (1996), each of which is incorporated herein by reference). As used herein, the term “library” means a collection of molecules. A library can contain a few or a large number of different molecules, varying from about ten molecules to several billion molecules or more. If desired, a molecule can be linked to a tag, which can facilitate recovery or identification of the molecule.

As used herein, the term “molecule” is used broadly to mean a polymeric or non-polymeric organic chemical such as a drug; a nucleic acid molecule such as an RNA, a cDNA or an oligonucleotide; a peptide, including a variant or modified peptide or peptide-like molecules, referred to herein as peptidomimetics, which mimic the activity of a peptide; or a protein such as an antibody or a growth factor receptor or a fragment thereof such as an Fv, Fd or Fab fragment of an antibody, which contains a binding domain. For convenience, the term “peptide” is used broadly herein to mean peptides, proteins, fragments of proteins and the like. A molecule also can be a non-naturally occurring molecule, which does not occur in nature, but is produced as a result of in vitro methods, or can be a naturally occurring molecule such as a protein or fragment thereof expressed from a cDNA library.

A tumor homing molecule also can be a peptidomimetic. As used herein, the term “peptidomimetic” is used broadly to mean a peptide-like molecule that has the binding activity of the tumor homing peptide. With respect to the tumor homing peptides of the invention, peptidomimetics, which include chemically modified peptides, peptide-like molecules containing non-naturally occurring amino acids, peptoids and the like, have the binding activity of a tumor homing peptide upon which the peptidomimetic is derived (see, for example, “Burger's Medicinal Chemistry and Drug Discovery,” supra, 1995).

Methods for identifying a peptidomimetic are well known in the art and include, for example, the screening of databases that contain libraries of potential peptidomimetics. For example, the Cambridge Structural Database contains a collection of greater than 300,000 compounds that have known crystal structures (Allen et al., Acta Crystallogr. Section B, 35:2331 (1979)). This structural depository is continually updated as new crystal structures are determined and can be screened for compounds having suitable shapes, for example, the same shape as a tumor homing molecule, as well as potential geometrical and chemical complementarity to a target molecule bound by a tumor homing peptide. Where no crystal structure of a tumor homing peptide or a target molecule that binds the tumor homing molecule is available, a structure can be generated using, for example, the program CONCORD (Rusinko et al., J. Chem. Inf. Comput. Sci. 29:251 (1989)). Another database, the Available Chemicals Directory (Molecular Design Limited, Informations Systems; San Leandro Calif.), contains about 100,000 compounds that are commercially available and also can be searched to identify potential peptidomimetics of a tumor homing molecule.

Methods for preparing libraries containing diverse populations of various types of molecules such as peptides, peptoids and peptidomimetics are well known in the art and various libraries are commercially available (see, for example, Ecker and Crooke, Biotechnology 13:351-360 (1995), and Blondelle et al., Trends Anal. Chem. 14:83-92 (1995), and the references cited therein, each of which is incorporated herein by reference; see, also, Goodman and Ro, Peptidomimetics for Drug Design, in “Burger's Medicinal Chemistry and Drug Discovery” Vol. 1 (ed. M. E. Wolff; John Wiley & Sons 1995), pages 803-861, and Gordon et al., J. Med. Chem. 37:1385-1401 (1994), each of which is incorporated herein by reference). Where a molecule is a peptide, protein or fragment thereof, the molecule can be produced in vitro directly or can be expressed from a nucleic acid, which can be produced in vitro. Methods of synthetic peptide and nucleic acid chemistry are well known in the art.

A library of molecules also can be produced, for example, by constructing a cDNA expression library from mRNA collected from a cell, tissue, organ or organism of interest. Methods for producing such libraries are well known in the art (see, for example, Sambrook et al., Molecular Cloning: A laboratory manual (Cold Spring Harbor Laboratory Press 1989), which is incorporated herein by reference). Preferably, a peptide encoded by the cDNA is expressed on the surface of a cell or a virus containing the cDNA. For example, cDNA can be cloned into a phage vector such as fuse 5 (Example IV), wherein, upon expression, the encoded peptide is expressed as a fusion protein on the surface of the phage.

In addition, a library of molecules can comprise a library of nucleic acid molecules, which can be DNA or RNA or an analog thereof. Nucleic acid molecules that bind, for example, to a cell surface receptor are well known (see, for example, O'Connell et al., Proc. Natl. Acad. Sci., USA 93:5883-5887 (1996); Tuerk and Gold, Science 249:505-510 (1990); Gold et al., Ann. Rev. Biochem. 64:763-797 (1995), each of which is incorporated herein by reference). Thus, a library of nucleic acid molecules can be administered to a subject having a tumor, and tumor homing molecules subsequently identified by in vivo panning. If desired, the nucleic acid molecules can be nucleic acid analogs that, for example, are less susceptible to attack by nucleases (see, for example, Jelinek et al., Biochemistry 34:11363-11372 (1995); Latham et al., Nucl. Acids Res. 22:2817-2822 (1994); Tam et al., Nucl. Acids Res. 22:977-986 (1994); Reed et al., Cancer Res. 59:6565-6570 (1990), each of which is incorporated herein by reference).

As set forth herein, in vivo panning can be used to identify a tumor homing molecule, which can be linked to an antimicrobial peptide to form a homing pro-apoptotic conjugate of the invention. In vivo panning comprises administering a library to a subject, collecting a sample of a tumor and identifying a tumor homing molecule. The presence of a tumor homing molecule can be identified using various methods well known in the art. Generally, the presence of a tumor homing molecule in a tumor is identified based on one or more characteristics common to the molecules present in the library, then the structure of a particular tumor homing molecule is identified. For example, a highly sensitive detection method such as mass spectrometry, either alone or in combination with a method such as gas chromatography, can be used to identify tumor homing molecules in a tumor. Thus, where a library comprises diverse molecules based generally on the structure of an organic molecule such as a drug, a tumor homing molecule can be identified by determining the presence of a parent peak for the particular molecule.

If desired, the tumor can be collected, then processed using a method such as HPLC, which can provide a fraction enriched in molecules having a defined range of molecular weights or polar or nonpolar characteristics or the like, depending, for example, on the general characteristics of the molecules comprising the library. Conditions for HPLC will depend on the chemistry of the particular molecule and are well known to those skilled in the art. Similarly, methods for bulk removal of potentially interfering cellular materials such as DNA, RNA, proteins, lipids or carbohydrates are well known in the art, as are methods for enriching a fraction containing an organic molecule using, for example, methods of selective extraction. Where a library comprises a population of diverse organic chemical molecules, each linked to a specific oligonucleotide tag, such that the specific molecule can be identified by determining the oligonucleotide sequence using polymerase chain reaction (PCR), genomic DNA can be removed from the sample of the collected tumor in order to reduce the potential for background PCR reactions. In addition, a library can comprise a population of diverse molecules such as organic chemical molecules, each linked to a common, shared tag. Based on the presence and properties of the shared tag, molecules of the library that selectively home to a tumor can be substantially isolated from a sample of the tumor. These and other methods can be useful for enriching a sample of a collected tumor for the particular tumor homing molecule, thereby removing potentially contaminating materials from the collected tumor sample and increasing the sensitivity of detecting a molecule.

Evidence provided herein indicates that a sufficient number of tumor homing molecules selectively homes to a tumor during in vivo panning such that the molecules readily can be identified. For example, various independent phage expressing the same peptide were identified in tumors formed from implanted human breast cancer cells (Table 2), from mouse melanoma cells (Table 3) or from human Kaposi's sarcoma cells (Table 4).

Although a substantial fraction of the identified tumor homing molecules have the same structure, the peptide inserts of only a small number of isolated phage were determined. It should be recognized, however, that hundreds of thousands to millions of phage expressing organ homing peptides have been recovered following in vivo pannings for organ homing molecules (see, for example, U.S. Pat. No. 5,622,699; Pasqualini and Ruoslahti, supra, 1996). These results indicate that specific tumor homing molecules will be present in substantial numbers in a tumor following in vivo homing, thereby increasing the ease with which the molecules can be identified.

Ease of identification of a tumor homing molecule, particularly an untagged molecule, depends on various factors, including the presence of potentially contaminating background cellular material. Thus, where the tumor homing molecule is an untagged peptide, a larger number must home to the tumor in order to identify the specific peptides against the background of cellular protein. In contrast, a much smaller number of an untagged organic chemical homing molecule such as a drug is identifiable because such molecules normally are absent from or present in only small numbers in the body. In such a case, a highly sensitive method such as mass spectrometry can be used to identify a tumor homing molecule. The skilled artisan will recognize that the method of identifying a molecule will depend, in part, on the chemistry of the particular molecule.

Where a tumor homing molecule is a nucleic acid molecule or is tagged with a nucleic acid molecule, an assay such as PCR can be particularly useful for identifying the presence of the molecule because, in principle, PCR can detect the presence of a single nucleic acid molecule (see, for example, Erlich, PCR Technology: Principles and Applications for DNA Amplification (Stockton Press 1989), which is incorporated herein by reference). Preliminary studies have demonstrated that, following intravenous injection of 10 ng of an approximately 6000 base pair plasmid into a mouse and 2 minutes in the circulation, the plasmid was detectable by PCR in a sample of lung. These results indicate that nucleic acid molecules are sufficiently stable when administered into the circulation such that in vivo panning can be used to identify nucleic acid molecules that selectively home to a tumor.

The molecules of a library can be tagged, which can facilitate recovery or identification of the molecule. As used herein, the term “tag” means a physical, chemical or biological moiety such as a plastic microbead, an oligonucleotide or a bacteriophage, respectively, that is linked to a molecule of the library. Methods for tagging a molecule are well known in the art (Hermanson, Bioconjugate Techniques (Academic Press 1996), which is incorporated herein by reference).

A tag, which can be a shared tag or a specific tag, can be useful for identifying the presence or structure of a tumor homing molecule of a library. As used herein, the term “shared tag” means a physical, chemical or biological moiety that is common to each molecule in a library. Biotin, for example, can be a shared tag that is linked to each molecule in a library. A shared tag can be useful to identify the presence of a molecule of the library in a sample and also can be useful to substantially isolate the molecules from a sample. For example, where the shared tag is biotin, the biotin-tagged molecules in a library can be substantially isolated by binding to streptavidin, or their presence can be identified by binding with a labeled streptavidin. Where a library is a phage display library, the phage that express the peptides are another example of a shared tag, since each peptide of the library is linked to a phage. In addition, a peptide such as the hemaglutinin antigen can be a shared tag that is linked to each molecule in a library, thereby allowing the use of an antibody specific for the hemaglutinin antigen to substantially isolate molecules of the library from a sample of a selected tumor.

A shared tag also can be a nucleic acid sequence that can be useful to identify the presence of molecules of the library in a sample or to substantially isolate molecules of a library from a sample. For example, each of the molecules of a library can be linked to the same selected nucleotide sequence, which constitutes the shared tag. An affinity column containing a nucleotide sequence that is complementary to the shared tag then can be used to hybridize molecules of the library containing the shared tag, thus substantially isolating the molecules from a tumor sample. A nucleotide sequence complementary to a portion of the shared nucleotide sequence tag also can be used as a PCR primer such that the presence of molecules containing the shared tag can be identified in a sample by PCR.

A tag also can be a specific tag. As used herein, the term “specific tag” means a physical, chemical or biological tag that is linked to a particular molecule in a library and is unique for that particular molecule. A specific tag is particularly useful if it is readily identifiable. A nucleotide sequence that is unique for a particular molecule of a library is an example of a specific tag. For example, the method of synthesizing peptides tagged with a unique nucleotide sequence provides a library of molecules, each containing a specific tag, such that upon determining the nucleotide sequence, the identity of the peptide is known (see Brenner and Lerner, Proc. Natl. Acad. Sci., USA 89:5381-5383 (1992), which is incorporated herein by reference). The use of a nucleotide sequence as a specific tag for a peptide or other type of molecule provides a simple means to identify the presence of the molecule in a sample because an extremely sensitive method such as PCR can be used to determine the nucleotide sequence of the specific tag, thereby identifying the sequence of the molecule linked thereto. Similarly, the nucleic acid sequence encoding a peptide expressed on a phage is another example of a specific tag, since sequencing of the specific tag identifies the amino acid sequence of the expressed peptide.

The presence of a shared tag or a specific tag can provide a means to identify or recover a tumor homing molecule following in vivo panning. In addition, the combination of a shared tag and specific tag can be particularly useful for identifying a tumor homing molecule. For example, a library of peptides can be prepared such that each is linked to a specific nucleotide sequence tag (see, for example, Brenner and Lerner, supra, 1992), wherein each specific nucleotide sequence tag has incorporated therein a shared tag such as biotin. Upon homing to a tumor, the particular tumor homing peptides can be substantially isolated from a sample of the tumor based on the shared tag and the specific peptides can be identified, for example, by PCR of the specific tag (see Erlich, supra, 1989).

A tag also can serve as a support. As used herein, the term “support” means a tag having a defined surface to which a molecule can be attached. In general, a tag useful as a support is a shared tag. For example, a support can be a biological tag such as a virus or virus-like particle such as a bacteriophage (“phage”); a bacterium such as E. coli; or a eukaryotic cell such as a yeast, insect or mammalian cell; or can be a physical tag such as a liposome or a microbead, which can be composed of a plastic, agarose, gelatin or other biological or inert material. If desired, a shared tag useful as a support can have linked thereto a specific tag. Thus, a phage display library, for example, can be considered to consist of the phage, which is a shared tag that also is a support, and the nucleic acid sequence encoding the expressed peptide, the nucleic acid sequence being a specific tag.

In general, a support should have a diameter less than about 10 μm to about 50 μm in its shortest dimension, such that the support can pass relatively unhindered through the capillary beds present in the subject and not occlude circulation. In addition, a support can be nontoxic, so that it does not perturb the normal expression of cell surface molecules or normal physiology of the subject, and biodegradable, particularly where the subject used for in vivo panning is not sacrificed to collect a selected tumor.

Where a molecule is linked to a support, the tagged molecule comprises the molecule attached to the surface of the support, such that the part of the molecule suspected of being able to interact with a target molecule in a cell in the subject is positioned so as to be able to participate in the interaction. For example, where the tumor homing molecule is suspected of being a ligand for a growth factor receptor, the binding portion of the molecule attached to a support is positioned so it can interact with the growth factor receptor on a cell in the tumor. If desired, an appropriate spacer molecule can be positioned between the molecule and the support such that the ability of the potential tumor homing molecule to interact with the target molecule is not hindered. A spacer molecule also can contain a reactive group, which provides a convenient and efficient means of linking a molecule to a support and, if desired, can contain a tag, which can facilitate recovery or identification of the molecule (see Hermanson, supra, 1996).

As exemplified herein, a peptide suspected of being able to home to a selected tumor such as a breast carcinoma or a melanoma was expressed as the N-terminus of a fusion protein, wherein the C-terminus consisted of a phage coat protein. Upon expression of the fusion protein, the C-terminal coat protein linked the fusion protein to the surface of a phage such that the N-terminal peptide was in a position to interact with a target molecule in the tumor. Thus, a molecule having a shared tag was formed by the linking of a peptide to a phage, wherein the phage provided a biological support, the peptide molecule was linked as a fusion protein, the phage-encoded portion of the fusion protein acted as a spacer molecule, and the nucleic acid encoding the peptide provided a specific tag allowing identification of a tumor homing peptide.

As used herein, the term “in vivo panning,” when used in reference to the identification of a tumor homing molecule, means a method of screening a library by administering the library to a subject and identifying a molecule that selectively homes to a tumor in the subject (see U.S. Pat. No. 5,622,699). The term “administering to a subject,” when used in reference to a library of molecules or a portion of such a library, is used in its broadest sense to mean that the library is delivered to a tumor in the subject, which, generally, is a vertebrate, particularly a mammal such as a human.

A library can be administered to a subject, for example, by injecting the library into the circulation of the subject such that the molecules pass through the tumor; after an appropriate period of time, circulation is terminated by sacrificing the subject or by removing a sample of the tumor (Example IV; see, also, U.S. Pat. No. 5,622,699; Pasqualini and Ruoslahti, supra, 1996). Alternatively, a cannula can be inserted into a blood vessel in the subject, such that the library is administered by perfusion for an appropriate period of time, after which the library can be removed from the circulation through the cannula or the subject can be sacrificed to collect the tumor, or the tumor can be sampled, to terminate circulation. Similarly, a library can be shunted through one or a few organs, including the tumor, by cannulation of the appropriate blood vessels in the subject. It is recognized that a library also can be administered to an isolated perfused tumor. Such panning in an isolated perfused tumor can be useful to identify molecules that bind to the tumor and, if desired, can be used as an initial screening of a library.

The use of in vivo panning to identify tumor homing molecules is exemplified herein by screening a phage peptide display library in tumor-bearing mice and identifying specific peptides that selectively homed to a breast tumor or to a melanoma tumor (Example IV). However, phage libraries that display protein receptor molecules, including, for example, an antibody or an antigen binding fragment of an antibody such an Fv, Fd or Fab fragment; a hormone receptor such as a growth factor receptor; or a cell adhesion receptor such as an integrin or a selectin also can be used to practice the invention. Variants of such molecules can be constructed using well known methods such as random mutagenesis, site-directed mutagenesis or codon based mutagenesis (see Huse, U.S. Pat. No. 5,264,563, issued Nov. 23, 1993, which is incorporated herein by reference). If desired, peptides can be chemically modified following expression of the phage but prior to administration to the subject. Thus, various types of phage display libraries can be screened by in vivo panning.

Phage display technology provides a means for expressing a diverse population of random or selectively randomized peptides. Various methods of phage display and methods for producing diverse populations of peptides are well known in the art. For example, Ladner et al. (U.S. Pat. No. 5,223,409, issued Jun. 29, 1993, which is incorporated herein by reference) describe methods for preparing diverse populations of binding domains on the surface of a phage. In particular, Ladner et al. describe phage vectors useful for producing a phage display library, as well as methods for selecting potential binding domains and producing randomly or selectively mutated binding domains.

Similarly, Smith and Scott (Meth. Enzymol. 217:228-257 (1993); see, also, Scott and Smith, Science 249: 386-390 (1990), each of which is incorporated herein by reference) describe methods of producing phage peptide display libraries, including vectors and methods of diversifying the population of peptides that are expressed (see, also, Huse, WO 91/07141 and WO 91/07149, each of which is incorporated herein by reference; see, also, Example IV). Phage display technology can be particularly powerful when used, for example, with a codon based mutagenesis method, which can be used to produce random peptides or randomly or desirably biased peptides (Huse, U.S. Pat. No. 5,264,563, supra, 1993). These or other well known methods can be used to produce a phage display library, which can be subjected to in vivo panning in order to identify tumor homing molecules useful in the homing pro-apoptotic conjugates of the invention.

In addition to screening a phage display library, in vivo panning can be used to screen various other types of libraries, including, for example, an RNA or DNA library or a chemical library. If desired, the tumor homing molecule can be tagged, which can facilitate recovery of the molecule from the tumor or identification of the molecule in the tumor. For example, where a library of organic molecules, each containing a shared tag, is screened, the tag can be a moiety such as biotin, which can be linked directly to the molecule or can be linked to a support containing the molecules. Biotin provides a shared tag useful for recovering the molecule from a selected tumor sample using an avidin or streptavidin affinity matrix. In addition, a molecule or a support containing a molecule can be linked to a hapten such as 4-ethoxy-methylene-2-phenyl-2-oxazoline-5-one (phOx), which can be bound by an anti-phOx antibody linked to a magnetic bead as a means to recover the molecule. Methods for purifying biotin or phOx labeled conjugates are known in the art and the materials for performing these procedures are commercially available (e.g., Invitrogen, La Jolla Calif.; and Promega Corp., Madison Wis.). In the case where a phage library is screened, the phage can be recovered using methods as disclosed in Example IV.

In vivo panning provides a method for directly identifying tumor homing molecules that can selectively home to a tumor. As used herein, the term “home” or “selectively home” means that a particular molecule binds relatively specifically to a target molecule present in the tumor following administration to a subject. In general, a tumor homing molecule is characterized, in part, by exhibiting at least a two-fold (2×) greater specific binding to a tumor as compared to a control organ or tissue.

It should be recognized that, in some cases, a molecule can localize nonspecifically to an organ or tissue containing a tumor. For example, in vivo panning of a phage display library can result in high background in organs such as liver and spleen, which contain a marked component of the reticuloendothelial system (RES). Thus, where a tumor is present, for example, in the liver, nonspecific binding of molecules due to uptake by the RES can make identifying a tumor homing molecule more difficult.

Selective homing of a tumor homing molecule can be distinguished from nonspecific binding, however, by detecting differences in the abilities of different individual phage to home to a tumor. For example, selective homing can be identified by combining a putative tumor homing molecule such as a peptide expressed on a phage with a large excess of non-infective phage or with about a five-fold excess of phage expressing unselected peptides, injecting the mixture into a subject and collecting a sample of the tumor. In the latter case, for example, provided the number of injected phage expressing tumor homing peptide is sufficiently low so as to be nonsaturating for the target molecule, a determination that greater than about 20% of the phage in the tumor express the putative tumor homing molecule is demonstrative evidence that the peptide expressed by the phage is a specific tumor homing molecule. In addition, nonspecific localization can be distinguished from selective homing by performing competition experiments using, for example, phage expressing a putative tumor homing peptide in combination with an excess amount of the “free” peptide (Example VII).

In addition, various methods can be used to prevent nonspecific binding of a molecule to an organ containing a component of the RES. For example, a molecule that homes selectively to a tumor present in an organ containing a component of the RES can be obtained by first blocking the RES using, for example, polystyrene latex particles or dextran sulfate (see Kalin et al., Nucl. Med. Biol. 20:171-174 (1993); Illum et al., J. Pharm. Sci. 75:16-22 (1986); Takeya et al., J. Gen. Microbiol. 100:373-379 (1977), each of which is incorporated herein by reference), then administering the library to the subject. For example, pre-administration of dextran sulfate 500 or polystyrene microspheres prior to administration of a test substance has been used to block nonspecific uptake of the test substance by Kupffer cells, which are the RES component of the liver (Illum et al., supra, 1986). Similarly, nonspecific uptake of agents by the RES has been blocked using carbon particles or silica (Takeya et al., supra, 1977) or a gelatine colloid (Kalin et al., supra, 1993). Thus, various agents useful for blocking nonspecific uptake by the RES are known and routinely used.

Nonspecific binding of phage to RES or to other sites also can be prevented by coinjecting, for example, mice with a specific phage display library together with the same phage made noninfective (Smith et al., supra, 1990, 1993). In addition, a peptide that homes to tumor in an organ containing an RES component can be identified by preparing a phage display library using phage that exhibit low background binding to the particular organ. For example, Merrill et al. (Proc. Natl. Acad. Sci., USA 93:3188-3192 (1996), which is incorporated herein by reference) selected lambda-type phage that are not taken up by the RES and, as a result, remain in the circulation for a prolonged period of time. A filamentous phage variant, for example, can be selected using similar methods.

Selective homing of a tumor homing molecule can be demonstrated by determining the specificity of a tumor homing molecule for the tumor as compared to a control organ or tissue. Selective homing also can be demonstrated by showing that molecules that home to a tumor, as identified by one round of in vivo panning, are enriched for tumor homing molecules in a subsequent round of in vivo panning. For example, phage expressing peptides that selectively home to a melanoma tumor were isolated by in vivo panning, then were subjected to additional rounds of in vivo panning. Following a second round of screening, phage recovered from the tumor showed a 3-fold enrichment in homing to the tumor as compared to brain. Phage recovered from the tumor after a third round of screening showed an average of 10-fold enrichment in homing to the tumor as compared to brain. Selective homing also can be demonstrated by showing that molecules that home to a selected tumor, as identified by one round of in vivo panning, are enriched for tumor homing molecules in a subsequent round of in vivo panning.

Tumor homing molecules can be identified by in vivo panning using, for example, a mouse containing a transplanted tumor. Such a transplanted tumor can be, for example, a human tumor that is transplanted into immunodeficient mice such as nude mice or a murine tumor that is maintained by passage in tissue culture or in mice. Due to the conserved nature of cellular receptors and of ligands that bind a particular receptor, it is expected that angiogenic vasculature and histologically similar tumor cells in various species can share common cell surface markers useful as target molecules for a tumor homing molecule. Thus, the skilled artisan would recognize that a tumor homing molecule identified using, for example, in vivo panning in a mouse having a murine tumor of a defined histological type such as a melanoma also would bind to the corresponding target molecule in a tumor in a human or other species. Similarly, tumors growing in experimental animals require associated neovascularization, just as that required for a tumor growing in a human or other species. Thus, a tumor homing molecule that binds a target molecule present in the vasculature in a tumor grown in a mouse likely also can bind to the corresponding target molecule in the vasculature of a tumor in a human or other mammalian subject. The general ability of a tumor homing molecule identified, for example, by homing to a human breast tumor, also to home to a human Kaposi's sarcoma or to a mouse melanoma indicates that the target molecules are shared by many tumors. Indeed, the results disclosed herein demonstrate that the target molecules are expressed in the neovasculature, which is host tissue (see Example VII).

A tumor homing molecule identified using in vivo panning in an experimental animal such as a mouse readily can be examined for the ability to bind to a corresponding tumor in a human patient by demonstrating, for example, that the molecule also can bind specifically to a sample of the tumor obtained from the patient. For example, the CDCRGDCFC (SEQ ID NO: 1) phage and NGR peptides have been shown to bind to blood vessels in microscopic sections of human tumors, whereas little or no binding occurs in the blood vessels of nontumor tissues. Thus, routine methods can be used to confirm that a tumor homing molecule identified using in vivo panning in an experimental animal also can bind the target molecule in a human tumor.

The steps of administering the library to the subject, collecting a selected tumor and identifying tumor homing molecules that home to the tumor, comprise a single round of in vivo panning. Although not required, one or more additional rounds of in vivo panning generally are performed. Where an additional round of in vivo panning is performed, the molecules recovered from the tumor in the previous round are administered to a subject, which can be the same subject used in the previous round, where only a part of the tumor was collected.

By performing a second round of in vivo panning, the relative binding selectivity of the molecules recovered from the first round can be determined by administering the identified molecules to a subject, collecting the tumor, and determining whether more phage are recovered from the tumor following the second round of screening as compared to those recovered following the first round. Although not required, a control organ or tissue also can be collected and the molecules recovered from the tumor can be compared with those recovered from the control organ. Ideally, no molecules are recovered from a control organ or tissue following a second or subsequent round of in vivo panning. Generally, however, a proportion of the molecules also will be present in a control organ or tissue. In this case, the ratio of molecules in the selected tumor as compared to the control organ (selected:control) can be determined. For example, phage that homed to melanoma following a first round of in vivo panning demonstrated a 3× enrichment in homing to the selected tumor as compared to the control organ, brain, following two additional rounds of panning (Example VIII).

Additional rounds of in vivo panning can be used to determine whether a particular molecule homes only to the selected tumor or can recognize a target on the tumor that also is expressed in one or more normal organs or tissues in a subject or is sufficiently similar to the target molecule on the tumor. It is unlikely that a tumor homing molecule also will home to a corresponding normal tissue because the method of in vivo panning selects only those molecules that home to the selected tumor. Where a tumor homing molecule also directs homing to one or more normal organs or tissues in addition to the tumor, the organs or tissues are considered to constitute a family of selected organs or tissues. Using the method of in vivo panning, molecules that home to only the selected tumor can be distinguished from molecules that also home to one or more selected organs or tissues. Such identification is expedited by collecting various organs or tissues during subsequent rounds of in vivo panning.

The term “control organ or tissue” is used to mean an organ or tissue other than the tumor for which the identification of a tumor homing molecule is desired. A control organ or tissue is characterized in that a tumor homing molecule does not selectively home to the control organ. A control organ or tissue can be collected, for example, to identify nonspecific binding of the molecule or to determine the selectivity of homing of the molecule. In addition, nonspecific binding can be identified by administering, for example, a control molecule, which is known not to home to a tumor but is chemically similar to a potential tumor homing molecule. Alternatively, where the administered molecules are linked to a support, administration of the supports, alone, also can be used to identify nonspecific binding. For example, a phage that expresses the gene III protein, alone, but that does not contain a peptide fusion protein, can be studied by in vivo panning to determine the level of nonspecific binding of the phage support.

As disclosed herein, specific homing of a tumor homing molecule readily can be identified by examining the selected tumor tissue as compared to a corresponding nontumor tissue, as well as to control organs or tissues. For example, immunohistological analysis can be performed on a tumor tissue and corresponding nontumor tissue using an antibody specific for a phage used to display tumor homing peptides (see Example VII). Alternatively, an antibody can be used that is specific for a shared tag that is expressed with the peptide, for example, a FLAG epitope or the like, such detection systems being commercially available.

In general, a library of molecules, which contains a diverse population of random or selectively randomized molecules of interest, is prepared, then administered to a subject. At a selected time after administration, the subject is sacrificed and the tumor is collected such that the molecules present in the tumor can be identified (see Example IV). If desired, one or more control organs or tissues or a part of a control organ or tissue can be sampled. For example, mice bearing a breast tumor or a melanoma tumor were injected with a phage peptide display library, then, after about 1 to 5 minutes, the mice were anesthetized, either frozen in liquid nitrogen or, preferably, are perfused through the heart to terminate circulation of the phage, the tumor and one or more control organs were collected from each, phage present in the tumor and the control organs were recovered and peptides that selectively homed to the respective tumors were identified (see Examples IV, V and VIII).

In the examples provided, the animals were sacrificed to collect the selected tumor and control organ or tissue. It should be recognized, however, that only a part of a tumor need be collected to recover a support containing a tumor homing molecule and, similarly, only part of an organ or tissue need be collected as a control. Thus, a part of a tumor, for example, can be collected by biopsy, such that a molecule such as a peptide expressed by a phage can be administered to the same subject a second time or more, as desired. Where the molecule that is to be administered a second time to the same subject is tagged or linked, for example, to a support, the tag or support should be nontoxic and biodegradable, so as not to interfere with subsequent rounds of screening.

In vitro screening of phage libraries previously has been used to identify peptides that bind to antibodies or to cell surface receptors (Smith and Scott, supra, 1993). For example, in vitro screening of phage peptide display libraries has been used to identify novel peptides that specifically bound to integrin adhesion receptors (Koivunen et al., J. Cell Biol. 124:373-380 (1994a), which is incorporated herein by reference) and to the human urokinase receptor (Goodson et al., Proc. Natl. Acad. Sci., USA 91:7129-7133 (1994)). However, such in vitro studies provide no insight as to whether a peptide that can specifically bind to a selected receptor in vitro also will bind the receptor in vivo or whether the binding peptide or the receptor are unique to a specific organ in the body. Furthermore, the in vitro methods are performed using defined, well-characterized target molecules in an artificial system. For example, Goodson et al., supra, 1994, utilized cells expressing a recombinant urokinase receptor. However, such in vitro methods are limited in that they require prior knowledge of the target molecule and yield little if any information regarding in vivo utility.

In vitro panning against cells in culture also has been used to identify molecules that can specifically bind to a receptor expressed by the cells (Barry et al., Nature Med. 2:299-305 (1996), which is incorporated herein by reference). However, the cell surface molecules that are expressed by a cell in vivo often change when the cell is grown in culture. Thus, in vitro panning methods using cells in culture also are limited in that there is no guarantee a molecule that is identified due to its binding to a cell in culture will have the same binding ability in vivo. Furthermore, it is not possible to use in vitro panning to distinguish molecules that home only to the tumor cells used in the screening, but not to other cell types.

In contrast, in vivo panning requires no prior knowledge or availability of a target molecule and identifies molecules that bind to cell surface target molecules that are expressed in vivo. Also, since the “nontargeted” tissues are present during the screening, the probability of isolating tumor homing molecules that lack specificity of homing is greatly reduced. Furthermore, in obtaining tumor homing molecules by in vivo panning, any molecules that may be particularly susceptible to degradation in the circulation in vivo due, for example, to a metabolic activity, are not recovered. Thus, in vivo panning provides significant advantages over previous methods by identifying tumor homing molecules that selectively home in vivo to a target molecule present in a tumor.

Although mechanisms by which the disclosed method of in vivo panning works have not been fully defined, one possibility is that a molecule such as a peptide expressed on a phage recognizes and binds to a target molecule present on endothelial cells lining the blood vessels in a tumor. Evidence indicates, for example, that the vascular tissues in various organs differ from one another and that such differences can be involved in regulating cellular trafficking in the body. For example, lymphocytes home to lymph nodes or other lymphoid tissues due, in part, to the expression of specific “address” molecules by the endothelial cells in those tissues (Salmi et al., Proc. Natl. Acad. Sci., USA 89:11436-11440 (1992); Springer, Cell 76:301-314 (1994)). Similarly, various leukocytes can recognize sites of inflammation due, in part, to the expression of endothelial cell markers induced by inflammatory signals (see Butcher and Picker, Science 272:60-66 (1996); Springer, supra, 1994). Thus, endothelial cell markers provide a potential target that can be selectively bound by a tumor homing molecule and used to direct a linked antimicrobial peptide to a tumor.

Additional components can be included as part of the homing pro-apoptotic conjugate, if desired. For example, in some cases, it can be desirable to utilize an oligopeptide spacer between a tumor homing molecule and the antimicrobial peptide. Such spacers are well known in the art, as described, for example, in Fitzpatrick and Garnett, Anticancer Drug Des. 10:1-9 (1995)).

A homing pro-apoptotic chimeric peptide of the invention can readily be synthesized in required quantities using routine methods of solid state peptide synthesis. A chimeric peptide of the invention also can be purchased from a commercial source (for example, AnaSpec, Inc.; San Jose, Calif.). Where an antimicrobial peptide is to be linked to a non-peptide tumor homing molecule, the antimicrobial peptide portion can be synthesized independently using well known methods or obtained from commercial sources.

Several methods can be used to link an antimicrobial peptide to a tumor homing molecule are known in the art, depending on the particular chemical characteristics of the molecule. For example, methods of linking haptens to carrier proteins as used routinely in the field of applied immunology (see, for example, Harlow and Lane, supra, 1988; Hermanson, supra, 1996).

A premade antimicrobial peptide also can be conjugated to a tumor homing peptide using, for example, carbodiimide conjugation (Bauminger and Wilchek, Meth. Enzymol. 70:151-159 (1980), which is incorporated herein by reference). Carbodiimides comprise a group of compounds that have the general formula R—N═C═N—R′, where R and R′ can be aliphatic or aromatic, and are used for synthesis of peptide bonds. The preparative procedure is simple, relatively fast, and is carried out under mild conditions. Carbodiimide compounds attack carboxylic groups to change them into reactive sites for free amino groups. Carbodiimide conjugation has been used to conjugate a variety of compounds to carriers for the production of antibodies.

The water soluble carbodiimide, 1-ethyl-3-(3-dimethylaminopropyl) carbodiimide (EDC) can be useful for conjugating an antimicrobial peptide to a tumor homing molecule. Such conjugation requires the presence of an amino group, which can be provided, for example, by an antimicrobial peptide, and a carboxyl group, which can be provided by the tumor homing molecule.

In addition to using carbodiimides for the direct formation of peptide bonds, EDC also can be used to prepare active esters such as N-hydroxysuccinimide (NHS) ester. The NHS ester, which binds only to amino groups, then can be used to induce the formation of an amide bond with the single amino group of the doxorubicin. The use of EDC and NHS in combination is commonly used for conjugation in order to increase yield of conjugate formation (Bauminger and Wilchek, supra, 1980).

Other methods for conjugating an antimicrobial peptide to a tumor homing molecule also can be used. For example, sodium periodate oxidation followed by reductive alkylation of appropriate reactants can be used, as can glutaraldehyde crosslinking. However, it is recognized that, regardless of which method of producing a conjugate of the invention is selected, a determination must be made that the tumor homing molecule maintains its targeting ability and that the antimicrobial peptide maintains its antimicrobial activity. Methods known in the art can confirm the activity of a homing pro-apoptotic conjugate of the invention.

The yield of antimicrobial peptide/tumor homing molecule conjugate formed is determined using routine methods. For example, HPLC or capillary electrophoresis or other qualitative or quantitative method can be used (see, for example, Liu et al., J. Chromatogr. 735:357-366 (1996); Rose et al., J. Chromatogr. 425:419-412 (1988), each of which is incorporated herein by reference; see, also, Example VIII). In particular, the skilled artisan will recognize that the choice of a method for determining yield of a conjugation reaction depends, in part, on the physical and chemical characteristics of the specific antimicrobial peptide and tumor homing molecule. Following conjugation, the reaction products are desalted to remove any free peptide or molecule.

The present invention also provides methods of directing an antimicrobial peptide in vivo to a tumor having angiogenic vasculature. The method is practiced by administering a homing pro-apoptotic conjugate of the invention to a subject containing a tumor having angiogenic vasculature. In a method of the invention for directing an antimicrobial peptide in vivo to a tumor having angiogenic vasculature, the antimicrobial peptide can include, for example, the sequence _{D}(KLAKLAK)₂. Particularly useful conjugates that can be administered to a subject containing a tumor having angiogenic vasculature include CNGRC-GG-_{D}(KLAKLAK)₂ and ACDCRGDCFC-GG-_{D}(KLAKLAK)₂.

The present invention additionally provides methods of inducing selective toxicity in vivo in a tumor having angiogenic vasculature. The methods are practiced by administering a homing pro-apoptotic conjugate of the invention to a subject containing a tumor having angiogenic vasculature. An antimicrobial peptide useful in inducing selective toxicity in a method of the invention can be, for example, a peptide containing the sequence _{D}(KLAKLAK)₂. Particularly useful conjugates that can be administered to induce selective toxicity in vivo in a tumor having angiogenic vasculature include CNGRC-GG-_{D}(KLAKLAK)₂ and ACDCRGDCFC-GG-_{D}(KLAKLAK)₂ Also provided herein are methods of treating a patient with a tumor having angiogenic vasculature. In such methods of treatment, a homing pro-apoptotic conjugate of the invention is administered to the patient and is selectively toxic to the tumor. The antimicrobial peptide portion can include, for example, the sequence _{D}(KLAKLAK)₂. In preferred embodiments, the homing pro-apoptotic conjugate has the sequence CNGRC-GG-_{D}(KLAKLAK)₂ or ACDCRGDCFC-GG-_{D}(KLAKLAK)₂.

When administered to a subject, a homing pro-apoptotic conjugate of the invention can be administered as a pharmaceutical composition containing, for example, the conjugate and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil or injectable organic esters.

A pharmaceutically acceptable carrier can contain physiologically acceptable compounds that act, for example, to stabilize or to increase the absorption of the conjugate. Such physiologically acceptable compounds include, for example, carbohydrates, such as glucose, sucrose or dextrans; antioxidants, such as ascorbic acid or glutathione; chelating agents; low molecular weight proteins; or other stabilizers or excipients. One skilled in the art would know that the choice of a pharmaceutically acceptable carrier, including a physiologically acceptable compound, depends, for example, on the route of administration of the composition. The pharmaceutical composition also can contain an agent such as a cancer therapeutic agent.

One skilled in the art would know that a homing pro-apoptotic conjugate of the invention can be administered as a pharmaceutical composition to a subject by various routes including, for example, orally or parenterally, such as intravenously. A pharmaceutical composition containing the conjugate can be administered by injection or by intubation. The pharmaceutical composition also can be a tumor homing molecule linked to liposomes or other polymer matrices, which can have incorporated therein, an antimicrobial peptide (Gregoriadis, Liposome Technology, Vol. 1 (CRC Press, Boca Raton, Fla. 1984), which is incorporated herein by reference). Liposomes, for example, which consist of phospholipids or other lipids, are nontoxic, physiologically acceptable and metabolizable carriers that are relatively simple to make and administer.

For the therapeutic methods disclosed herein, an effective amount of the homing pro-apoptotic conjugate must be administered to the subject. As used herein, the term “effective amount” means the amount of the conjugate that produces the desired effect. An effective amount often will depend on the particular antimicrobial peptide linked to the tumor homing molecule. An effective amount of a homing pro-apoptotic conjugate in which a tumor homing molecule is linked to a particular antimicrobial peptide can be determined using methods well known to those in the art.

The route of administration of a homing pro-apoptotic conjugate will depend, in part, on the chemical structure of the molecule. Peptides, for example, are not particularly useful when administered orally because they can be degraded in the digestive tract. However, methods for chemically modifying peptides to render them less susceptible to degradation by endogenous proteases or more absorbable through the alimentary tract, including incorporation of D-amino acids, are well known (see, for example, Blondelle et al., supra, 1995; Ecker and Crooke, supra, 1995; Goodman and Ro, supra, 1995). Such modifications can be performed on tumor homing peptides identified by in vivo panning as well as on antimicrobial peptides. In addition, methods for preparing libraries of peptidomimetics, which can contain D-amino acids, other non-naturally occurring amino acids, or chemically modified amino acids; or can be organic molecules that mimic the structure of a peptide; or can be peptoids such as vinylogous peptoids, are known in the art and can be used to identify tumor homing molecules that are stable for oral administration.

A tumor homing peptide can have a linear or cyclic structure. Cysteine residues were included in some peptides, allowing cyclization of the peptides. In particular, peptides containing at least two cysteine residues cyclize spontaneously. In addition, such cyclic peptides also can be active when present in a linear form (see, for example, Koivunen et al., supra, 1993). For example, the linear peptide, NGRAHA (SEQ ID NO: 6), also was useful as tumor homing molecule (see Table 2). Thus, in some cases one or more cysteine residues in the tumor homing peptides disclosed herein or otherwise identified as tumor homing peptides can be deleted without significantly affecting the tumor homing activity of the peptide. Methods for determining the necessity of a cysteine residue or of amino acid residues N-terminal or C-terminal to a cysteine residue for tumor homing activity of a peptide of the invention are routine and well known in the art.

As further disclosed herein, some, but not all, tumor homing molecules also can home to angiogenic vasculature that is not contained within a tumor. For example, tumor homing molecules containing either the RGD motif or the GSL motif specifically homed to retinal neovasculature (Smith et al., Invest. Ophthamol. Vis. Sci. 35:101-111 (1994), which is incorporated herein by reference), whereas tumor homing peptides containing the NGR motif did not accumulate substantially in this angiogenic vasculature. These results indicate that tumor vasculature expresses target molecules that are not substantially expressed by other kinds of angiogenic vasculature. Methods as disclosed herein can be used to distinguish tumor homing peptides from peptides that home to nontumor angiogenic vasculature. One skilled in the art understands that, preferably, for treatment of a tumor, one administers a conjugate having a tumor homing peptide, which selectively homes to tumor vasculature.

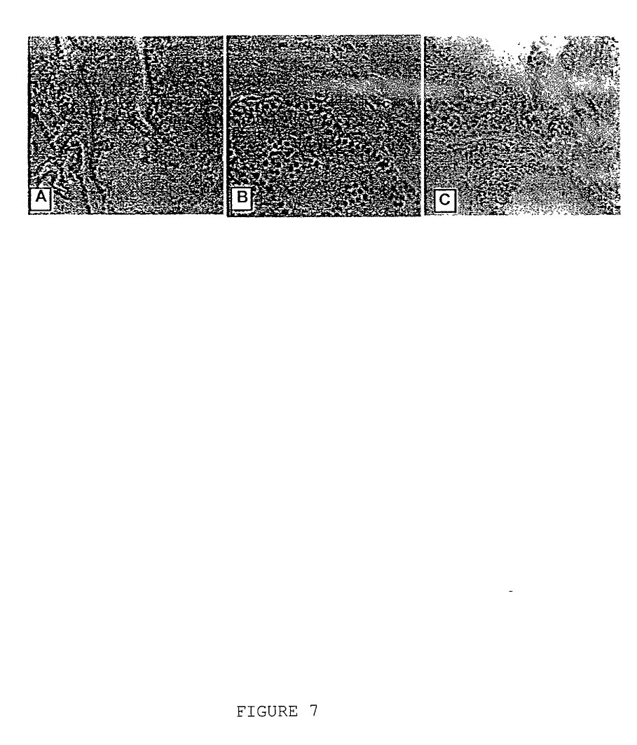

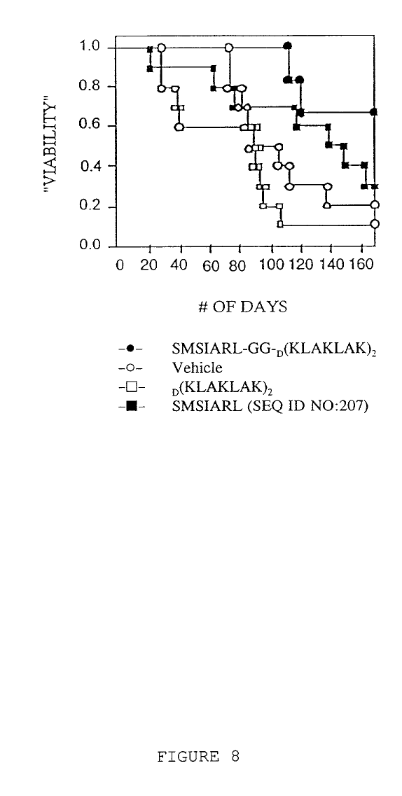

The invention provides chimeric prostate-homing pro-apoptotic peptides which can be used to treat, for example, benign prostate hyperplasia or cancer of the prostate. As disclosed herein, SMSIARL peptide (SEQ ID NO: 207) can selectively localize to prostate tissue, specifically prostate vasculature, when systemically administered (see Example IX.B and IX.E). Furthermore, the prostate homing peptide SMSIARL (SEQ ID NO: 207) can be used to selectively deliver a linked moiety, such as biotin or phage, to prostate tissue. As further disclosed herein, apoptosis was induced in mouse prostate by systemic administration of SMSIARL-GG-_{D}(KLAKLAK)₂ chimeric peptide; no evidence of apoptosis was observed in non-prostate tissues (see FIG. 7 and Example IX.C). The results disclosed herein also demonstrate that administration of SMSIARL-GG-_{D}(KLAKLAK)₂ chimeric peptide can extend the survival of TRAMP mice, which develop prostate cancer under the influence of a transgene as described in Gingrich et al., Cancer Res. 56: 4096-4102 (1996). FIG. 8 shows that SMSIARL-GG-_{D}(KLAKLAK)₂-treated mice survived longer than mice treated with vehicle alone, _{D}(KLAKLAK)₂ peptide alone, or SMSIARL peptide (SEQ ID NO: 207) alone. Based on these results, the invention provides a chimeric prostate-homing pro-apoptotic peptide as well as methods of using the peptide to treat a patient having prostate cancer as described further below.

Thus, the present invention provides a chimeric prostate-homing pro-apoptotic peptide that contains a prostate-homing peptide linked to an antimicrobial peptide, where the chimeric peptide is selectively internalized by prostate tissue and exhibits high toxicity thereto, while the antimicrobial peptide has low mammalian cell toxicity when not linked to the prostate-homing peptide. In a chimeric peptide of the invention, the prostate-homing peptide portion can contain, for example, the sequence SMSIARL (SEQ ID NO: 207) or a functionally equivalent sequence, and the antimicrobial peptide portion can have an amphipathic α-helical structure such as the sequence (KLAKLAK)₂ (SEQ ID NO: 200), (KLAKKLA)₂ (SEQ ID NO: 201), (KAAKKAA)₂ (SEQ ID NO: 202) or (KLGKKLG)₃ (SEQ ID NO: 203). In a preferred embodiment, the antimicrobial peptide portion contains the sequence _{D}(KLAKLAK)₂. An exemplary prostate-homing pro-apoptotic peptide is provided herein as SMSIARL-GG-_{D}(KLAKLAK)₂.

The present invention further provides a method of directing an antimicrobial peptide in vivo to a prostate cancer. The method includes the step of administering a chimeric prostate-homing pro-apoptotic peptide that contains a prostate-homing peptide linked to an antimicrobial peptide, where the chimeric peptide is selectively internalized by prostate tissue and exhibits high toxicity thereto, while the antimicrobial peptide has low mammalian cell toxicity when not linked to the prostate-homing peptide. In a method of the invention, the prostate-homing peptide can contain, for example, the sequence SMSIARL (SEQ ID NO: 207) or a functionally equivalent sequence, and the antimicrobial peptide can contain a sequence such as _{D}(KLAKLAK)₂. In a preferred embodiment, the chimeric prostate-homing pro-apoptotic peptide includes the sequence SMSIARL-GG-_{D}(KLAKLAK)₂. Also provided by the invention is a method of inducing selective toxicity in vivo in a prostate cancer. The method includes the step of administering to a subject containing a prostate cancer a chimeric prostate-homing pro-apoptotic peptide that contains a prostate-homing peptide linked to an antimicrobial peptide, where the chimeric peptide is selectively internalized by prostate tissue and exhibits high toxicity thereto, while the antimicrobial peptide has low mammalian cell toxicity when not linked to the prostate-homing peptide. The method of inducing selective toxicity in vivo in a prostate cancer can be practiced, for example, with a prostate-homing peptide containing the sequence SMSIARL (SEQ ID NO: 207) or a functionally equivalent sequence. The antimicrobial peptide can include, for example, the sequence _{D}(KLAKLAK)₂. In a preferred embodiment, the chimeric prostate-homing pro-apoptotic peptide includes the sequence SMSIARL-GG-_{D}(KLAKLAK)₂.

In addition, the invention provides a method of treating a patient having prostate cancer by administering to the patient a chimeric prostate-homing pro-apoptotic peptide of the invention, whereby the chimeric peptide is selectively toxic to the tumor. The chimeric peptide contains a prostate-homing peptide linked to an antimicrobial peptide, and the chimeric peptide is selectively internalized by prostate tissue and exhibits high toxicity thereto, while the antimicrobial peptide has low mammalian cell toxicity when not linked to the prostate-homing peptide. The prostate-homing peptide portion can contain, for example, the sequence SMSIARL (SEQ ID NO: 207) or a functionally equivalent sequence, and the antimicrobial peptide portion can contain, for example, the sequence _{D}(KLAKLAK)₂. In a preferred embodiment for treating a patient having a prostate tumor, the chimeric peptide contains the sequence SMSIARL-GG-_{D}(KLAKLAK)₂.

As used herein, the term “prostate-homing peptide” means a peptide that selectively homes in vivo to prostate tissue as compared to control tissue, such as brain. Such a peptide generally is characterized by at least a two-fold greater localization to prostatic tissue as compared to a control cell type or tissue. A prostate homing peptide can selectively home, for example, to prostate vasculature as compared to other cell types or other vasculature (see Example IX).

A chimeric peptide of the invention is selectively delivered to the prostate due to the selective homing activity of the prostate-homing peptide portion. A variety of prostate-homing peptides are useful in the invention, including SMSIARL (SEQ ID NO: 207) and VSFLEYR (SEQ ID NO: 222), which were identified by injection of an X₇ library into mice (Table 7) and subsequent in vivo panning as described in U.S. Pat. No. 5,622,699. The prostate homing peptides SMSIARL (SEQ ID NO: 21) and VSFLEYR (SEQ ID NO: 22) exhibited a 34-fold and 17-fold enrichment, respectively, in prostate as compared to brain.

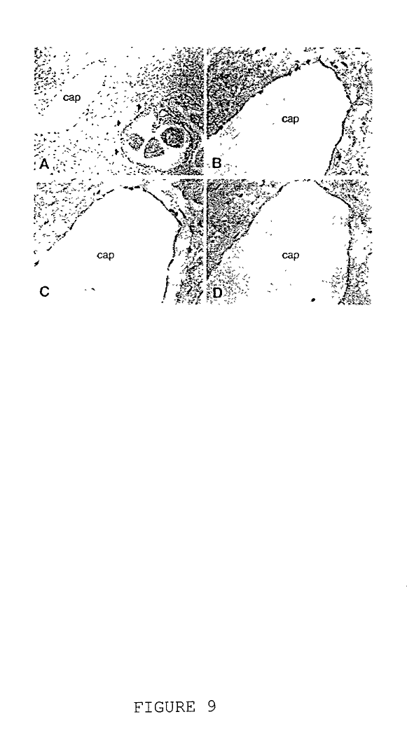

In one embodiment, the invention relies on a prostate-homing peptide which contains the sequence SMSIARL (SEQ ID NO: 207), or a functionally equivalent sequence. The term “functionally equivalent sequence,” as used herein in reference to the sequence SMSIARL (SEQ ID NO: 207), means a sequence that binds selectively to the endothelium of prostatic blood vessels, as shown in FIG. 9 for the sequence SMSIARL (SEQ ID NO: 207), and that functions similarly in that the sequence binds selectively to the same receptor.

It is understood that the chimeric prostate-homing pro-apoptotic peptides of the invention can be used to induce selective toxicity in a variety of prostatic disorders. Such disorders include benign nodular hyperplasia of the prostate as well as primary or secondary cancers including clinically apparent as well as subclinical cancers. Cancers to be treated with a chimeric peptide of the invention include prostatic carcinomas such as adenocarcinomas.

The following examples are intended to illustrate but not limit the present invention.

### EXAMPLE I

### Characterization of _{D}(KLAKLAK)₂

This example demonstrates that _{D}(KLAKLAK)₂ preferentially disrupts mitochondrial membrane and induces mitochondria-dependent apoptosis.

The synthetic 14-mer KLAKLAKKLAKLAK (SEQ ID NO: 200), designated (KLAKLAK)₂₁ was selected because it kills bacteria at a concentration two orders of magnitude lower than the concentration required to kill eukaryotic cells (Javadpour et al., J. Med. Chem. 39:3107-3113 (1996)). The all D-enantiomer, _{D}(KLAKLAK)₂, was used to avoid degradation by proteases (Bessalle et al., FEBS Lett. 274:151-155 (1990); Wade et al., Proc. Natl. Acad. Sci. 87:4761-4765 (1990)).

_{D}(KLAKLAK)₂ Preferentially Disrupts Mitochondrial Membranes

The ability of _{D}(KLAKLAK)₂ to disrupt mitochondrial membranes preferentially over eukaryotic plasma membranes was evaluated by mitochondrial swelling assays and in a mitochondria-dependent cell-free system of apoptosis, and by cytotoxicity assays.

Mitochondrial swelling assays were performed as follows. Briefly, rat liver mitochondria were prepared as described in Ellerby et al., J. Neurosci. 17:6165-6178 (1997). Peptides were synthesized at higher than 90% purity by HPLC (DLSLARLATARLAI (SEQ ID NO: 204), Coast Scientific, Inc., San Diego, Calif.; all other peptides, AnaSpec, Inc.). Mitochondria were treated with a concentration of 10 μM _{D}(KLAKLAK)₂, 10 μM DLSLARLATARLAI negative control peptide (SEQ ID NO: 204), or 200 μM CA⁺² as a positive control. Peptides were added to mitochondria in a cuvette, and swelling was quantified by measuring the optical absorbance at 520 nm.

As shown in FIG. 2a, 10 μM _{D}(KLAKLAK)₂ induced marked mitochondrial swelling. Mild swelling was evident at a concentration of 3 μM, two orders of magnitude less than the concentration required to kill eukaryotic cells (approximately 300 μM), as measured by the lethal concentration required to kill 50% of a cell monolayer (LC₅₀; Table 1). The non-α-helix forming peptide DLSLARLATARLAI (SEQ ID NO: 204) used as a negative control did not induce mitochondrial swelling. These results demonstrate that _{D}(KLAKLAK)₂ preferentially disrupts mitochondrial membranes as compared to eukaryotic plasma membranes.

TABLE 1
LC₅₀ (μM) FOR EUKARYOTIC CELLS TREATED
WITH HPP-l AND _{D}(KLAKLAK)₂

| | DMEC | | | | |
|---|---|---|---|---|---|
| | | Angiogenic | | | |
| | Angio- | | Cord | KS1767 | MDA-MB-435 |
| | static | Prolif. | Form | Prolif. | Prolif. |
| HPP-1 | 481 | 51 | 34 | 42 | 415 |
| _{D}(KLAKLAK)₂ | 492 | 346 | 368 | 387 | 333 |

Results are means of three independent experiments performed in triplicate (t-test, P < 0.03)

_{D}(KLAKLAK)₂ Induces Mitochondrial-dependent Apoptosis

The _{D}(KLAKLAK)₂ peptide was assayed for the ability to activate mitochondria-dependent apoptosis in a cell-free system composed of normal mitochondria suspended in normal cytosolic extract (Ellerby et al., J. Neurosci. 17:6165-6178 (1997)). Apoptosis was measured by characteristic caspase-3 processing from an inactive proform to the active protease (Alnemri et al., Cell 87:171 (1996)).

The cell-free apoptosis assays were performed essentially as follows. The cell-free systems were reconstituted as described in Ellerby et al., supra, 1997, and, for mitochondria-dependent reactions, rat liver mitochondria were suspended in normal (non-apoptotic) cytosolic extract prepared from dermal microvessel endothelial cells. After adding peptides at a concentration of 100 μM and incubating for 2 hours at 30° C. or 37° C., mitochondria were removed by centrifugation, and the supernatant analyzed by SDS/PAGE immunoblotting on a 12% gel (Biorad; Hercules, Calif.). Proteins were transferred to PVDF membranes (Biorad) and incubated with anti-caspase-3 antibody (Santa Cruz Biotechnology; Santa Cruz, Calif.), followed by ECL detection (Amersham; Arlington Heights, Ill.).

Characteristic caspase-3 processing was measured in dermal microvessel endothelial cell lysates as described in Ellerby et al., supra, 1997. Briefly, aliquots of cell lysates (1 μl lysate, 8-15 mg/ml) were added to 100 μM N-acetyl-Asp-Glu-Val-Asp-pNA (DEVD-pNA; BioMol; 100 μl, 100 mM HEPES, 10% sucrose, 0.1% CHAPS, 1 mM DTT, pH 7.0). Hydrolysis of DEVD-PNA was monitored spectrophotometrically (400 nm) at 25° C.

As shown in FIG. 2b, lane 4, characteristic caspase-3 processing the active protease was observed in the presence of mitochondria and _{D}(KLAKLAK)₂. The non-α-helix forming peptide DLSLARLATARLAI (SEQ ID NO: 204) used as a negative control was inactive when tested in the cell-free system and was not lethal to eukaryotic cells (FIG. 2b; see, also, Ellerby et al., supra, 1997).

In sum, these results indicate that _{D}(KLAKLAK)₂ preferentially disrupts mitochondrial membranes as compared to eukaryotic plasma membranes and activates mitochondria-dependent apoptosis.

### EXAMPLE II

### Characterization of HPP-1

This example demonstrates that CNGRC-GG-_{D}(KLAKLAK)₂ (HPP-1) inhibits angiogenesis in a tissue culture model.

Chimeras were prepared containing a homing domain linked through a glycinylglycine bridge to an antimicrobial peptide. As described above, peptides were synthesized commercially to higher than 90% purity by HPLC by AnaSpec, Inc.

The homing domain was either the cyclic (disulfide bond between cysteines) CNGRC peptide (SEQ ID NO: 8; see FIG. 1), or the double cyclic ACDCRGDCFC peptide (SEQ ID NO: 16), both of which have tumor-homing properties (Pasqualini et al., Nature Biotech. 15:542-546 (1997); Arap et al., Science 279:377-380 (1998)) and can be internalized (Arap et al., supra, 1998; Hart et al., J. Biol. Chem. 269:12468-12474 (1994); Bretscher et al., EMBO. J. 8:1341-1348 (1989)). Due to the presumed chiral nature of the homing domain-receptor interaction, the homing domain was synthesized from all L amino acids. The glycinylglycine bridge was used to couple the homing and antimicrobial domains to impart peptide flexibility and minimize potential steric interactions.

Viability of Dermal Microvessel Endothelial Cells Treated with HPP-1

Efficacy and specificity of HPP-1 were evaluated in a tissue culture model of angiogenesis as described in Goto et al., Lab. Invest. 69:508-517 (1993). During angiogenesis, capillary endothelial cells proliferate and migrate (Risau, Nature 386:671-674 (1997); Zetter, Ann. Rev. Med. 49:407-424 (1998)). Cord formation is a form of migration that is represented in vitro by a change in endothelial cell morphology from the usual “cobblestones” to chains or cords of cells as shown in FIG. 3 (see, also, Goto et al., supra, 1993).

The effect of HPP-1 was assayed on normal human dermal microvessel endothelial cells (DMECs) under the angiogenic conditions of proliferation and cord formation. In addition, the effect of HPP-1 was assayed under the angiostatic condition of a monolayer maintained at 100% confluency (as described below).

Briefly, dermal microvessel endothelial cells (DMECs) were grown in CADMEC Growth Media™ (Cell Applications, Inc.; San Diego, Calif.). Dermal microvessel endothelial cells were then cultured under three experimental conditions: proliferation, 30% confluency in a growth media supplemented with 500 ng/ml human recombinant vascular endothelial growth factor (VEGF; Pharmingen); no proliferation, 100% confluency in media formulated to maintain a monolayer; and cord formation, 60% confluency (required for induction) in media formulated to induce cord formation. The KS1767 and MDA-MB-435 cells were cultured as described in Arap et al., supra, 1998; Hernier et al., supra, 1994).

Percent viability and LC₅₀ (Table 1) were determined by apoptotic morphology as described in Ellerby et al., J. Neurosci. 17:6165-6178 (1997). For the percent viability assays, dermal microvessel endothelial cells were treated with 60 μM HPP-1 or control peptide _{D}(KLAKLAK)₂. At the indicated time points, cell culture medium was aspirated from adherent cells, and the cells were gently washed once with PBS at 37° C. Subsequently, a 20-fold dilution of the dye mixture (100 μg/ml acridine orange and 100 μg/ml ethidium bromide) in PBS was gently pipetted on the cells, which were viewed on an inverted microscope (Nikon TE 300). Cells with nuclei exhibiting margination and condensation of the chromatin and/or nuclear fragmentation (early/mid apoptosis) or with compromised plasma membranes (late apoptosis) were scored as not viable. At least 500 cells were scored for a given time point in each experiment. Percent viability was calculated relative to untreated controls. The LC₅₀ for monolayer, proliferation (60% confluency), and cord formation were scored at 72 hours.

As shown in FIG. 3, treatment of dermal microvessel endothelial cells with 60 mM HPP-1 led to a decrease in percent viability with time relative to untreated controls, under the conditions of proliferation or cord formation (see FIGS. 3c and 3d, respectively). In contrast, treatment with the untargeted peptide _{D}(KLAKLAK)₂ as a negative control led to a negligible loss in viability. Furthermore, the LC₅₀ for proliferating or migrating dermal microvessel endothelial cells treated with HPP-1 was an order of magnitude lower than the LC₅₀ for angiostatic dermal microvessel endothelial cells maintained in a monolayer at 100% confluency (Table 1). These results demonstrate preferential killing by HPP-1 under angiogenic conditions as compared to angiostatic conditions.

Various controls also were assayed for an effect on viability of dermal microvessel endothelial cells. The LC₅₀ for the untargeted control _{D}(KLAKLAK)₂ under angiogenic conditions was similar to the LC₅₀ for HPP-1 under angiostatic conditions. Furthermore, a mixture of uncoupled _{D}(KLAKLAK)₂ and CNGRC (SEQ ID NO: 8), a non-targeted form CARAC-GG-_{D}(KLAKLAK)₂ and a scrambled form CGRNC-GG-_{D}(KLAKLAK)₂ gave results similar to _{D}(KLAKLAK) ₂. Moreover, the alternative prototype ACDCRGDCFC-GG-_{D}(KLAKLAK)₂ gave results similar to those for CNGRC-GG-_{D}(KLAKLAK)₂ (HPP-1; data not shown).

Mitochondrial Morpholoay of Dermal Microvessel Endothelial Cells Treated with HPP-1

Mitochondrial morphology of dermal microvessel endothelial cells was assessed in proliferating cells after treatment with 60 μM HPP-1, ACDCRGDCFC-GG-_{D}(KLAKLAK)₂ or untargeted _{D}(KLAKLAK)₂ as follows. Dermal microvessel endothelial cells were stained after 24 and 72 hours treatment with peptide by incubation for 30 min at 37° C. with 100 nM mitochondrial stain MitoTracker Red™ (Molecular Probes, Inc., Eugene Oreg.) and 500 nM of the nuclear stain DAPI (Molecular Probes, Inc.). Mitochondria were subsequently visualized using fluorescence microscopy (100×) under an inverted microscope using a triple wavelength filter set (Nikon).

The mitochondria of dermal microvessel endothelial cells treated for 24 hours with _{D}(KLAKLAK)₂ remained morphologically normal, while those treated with each prototype exhibited altered mitochondrial morphology. In particular, altered mitochondrial morphology was evident in approximately 80% of cells treated with CNGRC-GG-_{D}(KLAKLAK)₂ or ACDCRGDCFC-GG-_{D}(KLAKLAK)₂ before the cells rounded-up. Ultimately, the CNGRC-GG-_{D}(KLAKLAK)₂ (HPP-1) treated dermal microvessel endothelial cells exhibited the classic morphological indicators of apoptosis, including nuclear condensation and fragmentation, as seen at 72 hours (Ellerby et al., supra, 1997). Apoptotic cell death was confirmed with by assaying for caspase activity (see FIG. 3b; Ellerby et al., supra, 1997).

Viability of KS1767 and MDA-MB-435 Cells Treated with HPP-1

The chimeric HPP-1 peptide was as toxic to KS1767 cells, which are derived from Kaposi's sarcoma, as to the proliferating or migrating dermal microvessel endothelial cells (Table 1; Hernier et al., AIDS 8: 575-581 (1994)). In contrast, HPP-1 was less toxic to MDA-MB-435 human breast carcinoma cells by approximately one order of magnitude (Table 1). KS1767 cells, which are of endothelial origin, resemble angiogenic endothelial cells and bind the CNGRC peptide (SEQ ID NO: 8), whereas MDA-MB-435 cells do not (Samaniego et al., Amer. J. Path. 152:1433-1443 (1998); Arap et al., supra, 1998).

In sum, these results demonstrate that HPP-1 induces mitochondrial swelling and apoptosis in dermal microvessel endothelial cells.

### EXAMPLE III

### in vivo Activity of Homing Pro-apoptotic Peptides

This example demonstrates that CNGRC-GG-_{D}(KLAKLAK)₂ (HPP-1) inhibits tumor growth and prolongs survival of tumor bearing animals. This example further demonstrates that CDCRGDCFC-GG-_{D}(KLAKLAK)₂ inhibits retinal neovascularization.

A. In vivo Activity of CNGRC-GG-_{D}(KLAKLAK)₂ (HPP-1)

The activity of HPP-1 was tested in vivo using nude mice bearing human MDA-MD-435 breast carcinoma xenografts as follows. Briefly, MDA-MB-435 and C8161-derived tumor xenografts were established in 2 month-old female nude mice (Jackson Labs; Bar Harbor, Me.) as described in Arap et al., supra, 1998. After mice were anesthetized with a mixture of 2,2,2-tribromoethanol (Aldrich; Milwaukee, Wis.) and 2-methyl-butanol (Aldrich) prepared in distilled water, peptides were administered intravenously through the tail vein at a dose of 250 μg/week/mouse given slowly in a volume of 200 μl. Three-dimensional measurements of tumors were taken by caliper under anesthesia and used to calculate tumor volume (Pasqualini et al., supra, 1996).

As shown in FIG. 4a, tumor volume was smaller, on average by one order of magnitude, than in controls, which were a non-targeted CARAC-GG-_{D}(KLAKLAK)₂ and a mixture of uncoupled _{D}(KLAKLAK)₂ and CNGRC (SEQ ID NO: 8) peptides. As further shown in FIG. 4b, survival was longer in the HPP-1 treated groups than in control groups. Some of the HPP-1 treated mice outlived control mice by several months, indicating that both primary tumor growth and metastasis were inhibited by HPP-1. Histopathological analysis revealed pronounced destruction of tumor architecture and widespread cell death in the tumors, demonstrating that the cell death was approximately equally apoptotic and necrotic. In similar experiments, HPP-1 was also effective against tumors derived from the human melanoma cell line C8161 (Welch et al., Int. J. Cancer 47:227-237 (1991)).

These results indicate that homing pro-apoptotic conjugates such as CNGRC-GG-_{D}(KLAKLAK)₂ (HPP-1) have strong anti-tumor activities in vivo.

B. in vivo Activity of CDCRGDCFC-GG-_{D}(KLAKLAK)₂

Retinal angiogenesis was oxygen-induced in newborn mice. Mice were subsequently treated with a single 13 μg intravenous dose (one animal per group) of vehicle; CDCRGDCFC-GG-_{D}(KLAKLAK)2; or a control mixture of unconjugated CDCRGDCFC (SEQ ID NO: 1) and _{D}(KLAKLAK)₂. Four days later, retinal neovessel number was determined for each treatment.

The results shown in FIG. 5 demonstrated that the number of retinal neovessels was reduced in mice treated with the homing pro-apoptotic peptide CDCRGDCFC-GG-_{D}(KLAKLAK)₂ (column 2; striped bar) as compared to mice treated with vehicle alone (column 1; black bar) or with untargeted pro-apoptotic peptide (column 3; hatched bar). In particular, the angiogenic response in mice treated with the homing pro-apoptotic peptide CDCRGDCFC-GG-_{D}(KLAKLAK)₂ was only 30-40% of the response seen in the control mice.

These results show that a homing pro-apoptotic peptide such as CDCRGDCFC-GG-_{D}(KLAKLAK)₂ can selectively inhibit an angiogenic response such as retinal neovascularization.

### EXAMPLE IV

### in vivo Panning

This example demonstrates methods for preparing a phage library and screening the library using in vivo panning to identify phage expressing tumor homing peptides.

A. Preparation of Phage Libraries:

Phage display libraries were constructed using the fuse 5 vector as described by Koivunen et al. (supra, 1995; Koivunen et al., supra, 1994b). Libraries encoding peptides designated CXSC (SEQ ID NO: 9), CX₆C (SEQ ID NO: 10), CX₇C (SEQ ID NO: 11) and CX₃CX₃CX₃C (SEQ ID NO: 12) were prepared, where “C” indicates cysteine and “X_{N}” indicates the given number of individually selected amino acids. These libraries can display cyclic peptides when at least two cysteine residues are present in the peptide. In addition, a library that did not contain defined cysteine residues also was constructed. Such a library results in the production primarily of linear peptides, although cyclic peptides also can occur due to random probability.

A biased library based on the sequence CXXXNGRXX (SEQ ID NO: 13) also was constructed. Furthermore, in some cases the CXXXNGRXX (SEQ ID NO: 13) library was further biased by in the incorporation of cysteine residues flanking the NGR sequence, i.e., CXXCNGRCX (SEQ ID NO: 14; see Table 2).

The libraries containing the defined cysteine residues were generated using oligonucleotides constructed such that “C” was encoded by the codon TGT and “XN” was encoded by NNK, where “N” is equal molar mixtures of A, C, G and T, and where “K” is equal molar mixtures of G and T. Thus, the peptide represented by CX₅C (SEQ ID NO: 9) can be represented by an oligonucleotide having the sequence TGT(NNK)₅TGT (SEQ ID NO: 14). Oligonucleotides were made double stranded by 3 cycles of PCR amplification, purified and ligated to the nucleic acid encoding the gene III protein in the fuse 5 vector such that, upon expression, the peptide is present as a fusion protein at the N-terminus of the gene III protein.

The vectors were transfected by electroporation into MC1061 cells. Bacteria were cultured for 24 hr in the presence of 20 μg/ml tetracycline, then phage were collected from the supernatant by precipitation twice using polyethylene glycol. Each library contained about 5×10⁹ to 5×10¹⁴ transducing units (TU; individual recombinant phage).

B. in vivo Panning of Phage:

Tumors were transplanted into mice as described in Examples V and VI, below. A mixture of phage libraries containing 1×10⁹ to 1×10¹⁴ TU was diluted in 200 μl DMEM and injected into the tail vein of anesthetized mice (AVERTIN (0.015 ml/g); see U.S. Pat. No. 5,622,699; Pasqualini and Ruoslahti, supra, 1996). After 1-4 minutes, mice were snap frozen in liquid nitrogen. To recover the phage, carcasses were partially thawed at room temperature for 1 hr, tumors and control organs were collected and weighed, then were ground in 1 ml DMEM-PI (DMEM containing protease inhibitors (PI); phenylmethylsulfonyl fluoride (PMSF; 1 mM), aprotinin (20 μg/ml), leupeptin (1 μg/ml)).

Alternatively, following introduction of a library into a mouse, circulation of the library is terminated by perfusion through the heart. Briefly, mice were anesthetized with AVERTIN, then the heart was exposed and a 0.4 mm needle connected through a 0.5 mm cannula to a 10 cc syringe was inserted into the left ventricle. An incision was made on the right atrium and 5 to 10 ml of DMEM was slowly administered, perfusing the whole body over about a 5 to 10 min period. Efficiency of the perfusion was monitored directly by histologic analysis.

Tumor and organ samples were washed 3 times with ice cold DMEM-PI containing 1% bovine serum albumin (BSA), then directly incubated with 1 ml K91-kan bacteria for 1 hr. Ten ml NZY medium containing 0.2 μg/ml tetracycline (NZY/tet) was added to the bacterial culture, the mixture was incubated in a 37° C. shaker for 1 hr, then 10 μl or 100 μl aliquots were plated in agar plates containing 12.5 μg/ml tetracycline (tet/agar).

Individual colonies containing phage recovered from a tumor were grown for 16 hr in 5 ml NZY/tet. The bacterial cultures obtained from the individual colonies were pooled and the phage were purified and re-injected into mice as described above for a second round of in vivo panning. In general, a third round of panning also was performed. Phage DNA was purified from individual bacterial colonies obtained from the final round of in vivo panning and the DNA sequences encoding the peptides expressed by selected phage were determined (see Koivunen et al., supra, 1994b).

### EXAMPLE V

### Identification of Tumor Homing Peptides by in vivo Panning against a Breast Tumor

This example demonstrates that in vivo panning can be performed against a breast tumor to identify tumor homing peptides that home to various tumors.

Human 435 breast carcinoma cells (Price et al., Cancer Res. 50:717-721 (1990)) were inoculated into the mammary fat pad of nude mice. When the tumors attained a diameter of about 1 cm, either a phage targeting experiment was performed, in which phage expressing a specific peptide were administered to the tumor bearing mouse, or in vivo panning was performed.

The breast tumor bearing mice were injected with 1×10⁹ phage expressing a library of CX₃CX₃CX₃C (SEQ ID NO: 12) peptides, where X₃ indicates three groups of independently selected, random amino acids. The phage were allowed to circulate for 4 min, then the mice were anesthetized, snap frozen in liquid nitrogen while under anesthesia, and the tumor was removed. Phage were isolated from the tumor and subjected to two additional rounds of in vivo panning.

TABLE 2
PEPTIDES FROM PHAGE RECOVERED
FROM HUMAN BREAST CANCER

| | |
|---|---|
| CGRECPRLCQSSC (2*) | CNGRCVSGCAGRC (3) |
| CGEACGGQCALPC (20) | TWSGYGVYW (21) |
| PSCAYMCIT (22) | WESLYFPRE (23) |
| SKVLYYNWE (24) | CGLMCQGACFDVC (25) |
| CERACRNLCREGC (26) | CPRGCLAVCVSQC (27) |

| | | |
|---|---|---|
| CKVCNGRCCC (23) | CEMCNGRCMC (29) | CPLCNGRCAL (30) |
| CPTCNGRCVR (31) | CGVCNGRCGL (32) | CEQCNGRCGQ (33) |
| CRNCNGRCEG (34) | CVLCNGRCWS (35) | CVTCNGRCRV (36) |
| CTECNGRCQL (37) | CRTCNGRCLE (38) | CETCNGRCVG (39) |
| CAVCNGRCGF (40) | CRDLNGRKVM (41) | CSCCNCRCGD (42) |
| CWGCNGRCRM (43) | CPLCNGRCAR (44) | CKSCNGRCLA (45) |
| CVPCNGRCHE (46) | CQSCNCRCVR (47) | CRTCNGRCQV (48) |
| CVQCNGRCAL (49) | CRCCNGRCSP (50) | CASNNGRVVL (51) |
| CGRCNCRCLL (52) | CWLCNGRCGR (53) | CSKCNCRCGH (54) |
| CVWCNGRCGL (55) | CIRCNGRCSV (56) | CGECNGRCVE (57) |
| CEGVNGRRLR (58) | CLSCNGRCPS (59) | CEVCNGRCAL (60) |
| CGSLVRC (5) | GRSQMQT (61) | HHTRFVS (62) |
| SKCLRHR (63) | VASVSVA (64) | WRVLAAF (65) |
| KMGPKVW (66) | TFSGSRE (67) | SPGSWTW (68) |
| NPRWFWD (69) | GRWYKWA (70) | IKARASP (71) |
| SGWCYRC (72) | ALVGLMR (73) | LWAEMTG (74) |
| CWSGVDC (75) | DTLRLRI (76) | SKSSCVS (77) |
| IVADYQR (78) | VWRTGHL (79) | VVDRFPD (80) |
| LSMFTRP (81) | GLPVKWS (82) | TMYPGWL (83) |
| CVMVRDGDC (84) | CVRTRPC (85) | CQLAAVC (86) |
| CGVGSSC (87) | CVSCPRC (88) | CCLSDSC (89) |
| CGEGHPC (90) | CYTADPC (91) | CELSLISKC (92) |
| CPEHRSLVC (93) | CLVVHEAAC (94) | CYVELHC (95) |
| CWRKFYC (96) | CFWPNRC (97) | CYSYFLAC (98) |
| CPRCSRC (99) | CRLGIAC (100) | CDDSWKC (101) |
| CAQLLQVSC (102) | CYPADPC (103) | CKALSQAC (104) |
| CTDYVRC (105) | CGETMRC (106) | |

* - numbers in parentheses indicate SEQ ID NO:.

Following the third round of panning, phage were quantitated and the peptide sequences expressed by the cloned phage were determined. The cloned phage expressed various different peptides, including those shown in Table 2. Similarly, CX₇C (SEQ ID NO: 11) and CX₅C (SEQ ID NO: 9) libraries were screened and breast tumor homing peptides were identified (Table 2). These results demonstrate that in vivo panning against a breast tumor can identify tumor homing molecules.

### EXAMPLE VI

### in vivo Targeting of a Phage Expressing an an RGD Peptide to a Tumor

Human 435 breast carcinoma cells were inoculated into the mammary fat pad of nude mice. When the tumors attained a diameter of about 1 cm, phage expressing a specific RGD-containing peptide were administered to the tumor bearing mouse. Similar results to those discussed below also were obtained with nude mice bearing tumors formed by implantation of human melanoma C8161 cells or by implantation of mouse B16 melanoma cells.

1×10⁹ phage expressing the RGD-containing peptide, CDCRGDCFC (SEQ ID NO: 1; see, Koivunen et al., supra, 1995) or control (insertless) phage were injected intravenously (iv) into the mice and allowed to circulate for 4 min. The mice then were snap frozen or perfused through the heart while under anesthesia, and various organs, including tumor, brain and kidney, were removed and the phage present in the organs was quantitated (see U.S. Pat. No. 5,622,699; Pasqualini and Ruoslahti, supra, 1996).

Approximately 2-3 times more phage expressing the CDCRGDCFC (SEQ ID NO: 1) peptide were detected in the breast tumor as compared to brain and kidney, indicating the CDCRGDCFC (SEQ ID NO: 1; RGD phage) peptide resulted in selective homing of the phage to the breast tumor. In a parallel study, unselected phage, which express various, diverse peptides, were injected into tumor-bearing mice and various organs were examined for the presence of phage. Far more phage were present in kidney and, to a lesser extent, brain, as compared to the tumor. Thus, the 80-fold more RGD-expressing phage than unselected phage concentrated in the tumor. These results indicate that phage expressing the RGD-containing peptide home to a tumor, possibly due to the expression of the αᵥβ₃ integrin on blood vessels forming in the tumor.

Specificity of the breast tumor homing peptide was demonstrated by competition experiments, in which coinjection of 500 μg free peptide, ACDCRGDCFCG (SEQ ID NO: 16; see Pasqualini et al., supra, 1997) with the phage expressing the tumor homing peptide reduced the amount of phage in the tumor by about tenfold, whereas coinjection with the inactive control peptide, GRGESP (SEQ ID NO: 17) essentially had no effect. These results demonstrate that phage displaying a peptide that can bind to an integrin expressed on angiogenic vasculature can selectively home in vivo to an organ or tissue such as a tumor containing such vasculature.

### EXAMPLE VII

### Immunohistologic Analysis of Tumor Homing Peptides

This example provides a method of identifying the localization of tumor homing molecules by immunohistologic examination.

Localization of phage expressing a tumor homing peptide was identified by immunochemical methods in histologic sections obtained either 5 min or 24 hr after administration of phage expressing a tumor homing peptide (“peptide-phage”) to a tumor bearing mouse. For samples obtained 5 min following administration of the peptide-phage, mice were perfused with DMEM and various organs, including the tumor, were removed and fixed in Bouin's solution. For samples obtained at 24 hr, no peptide-phage remains in the circulation and, therefore, perfusion was not required. Histologic sections were prepared and reacted with anti-M13 (phage) antibodies (Pharmacia Biotech; see U.S. Pat. No. 5,622,699; Pasqualini and Ruoslahti, supra, 1996). Visualization of the bound anti-M13 antibody was performed using a peroxidase-conjugated second antibody (Sigma; St. Louis Mo.) according to the manufacturer's instructions.

As discussed in Example VI, phage expressing the tumor homing peptide, CDCRGDCFC (SEQ ID NO: 1; “RGD phage), were administered intravenously to mice bearing the breast tumor. In addition, the RGD phage were administered to mice bearing a mouse melanoma or a human Kaposi's sarcoma. Circulation of the phage was terminated and mice were sacrificed as described above and samples of the tumor and of skin adjacent to the tumor, brain, kidney, lung and liver were collected. Immunohistochemical staining for the phage showed accumulation of the RGD phage in the blood vessels present in the breast tumor as well as in the melanoma and the Kaposi's sarcoma, whereas little or no staining was observed in the control organs.

Similar experiments were performed using phage expressing the tumor homing peptide, CNGRCVSGCAGRC (SEQ ID NO: 3; “NGR phage”), which was identified by in vivo panning against a tumor formed by the MDA-MB-435 breast carcinoma. In these experiments, NGR phage or control phage, which do not express a peptide, were administered to mice bearing tumors formed by the MDA-MB-435 breast carcinoma or by a human SLK Kaposi's sarcoma xenograft, then the mice were sacrificed as described above and tumors were collected as well as control organs, including brain, lymph node, kidney, pancreas, uterus, mammary fat pad, lung, intestine, skin, skeletal muscle, heart and epithelium of the renal calices, bladder and ureter. Histological samples were prepared and examined by immunostaining as described above.

In samples obtained from mice sacrificed 4 min after administration of the NGR phage, immunostaining of the vasculature of both the breast tumor and the Kaposi's sarcoma was observed. Very little or no staining was observed in the endothelium of the these tumors in mice administered an insertless control phage. In the samples obtained from mice sacrificed 24 hr after administration of the NGR phage, staining of the tumor samples appeared to have spread outside of the vessels, into the breast tumor parenchyma and the Kaposi's sarcoma parenchyma. Again, little or no staining was observed in samples prepared from these tumors in mice administered the insertless control phage. In addition, little or no staining was observed in various control organs in mice administered the NGR phage.

In other experiments, similar results were obtained following administration of phage expressing the NGR tumor homing peptides, NGRAHA (SEQ ID NO: 6) or CVLNGRMEC (SEQ ID NO: 7), to tumor bearing mice. Also, as discussed below, similar results were obtained using phage expressing the GSL tumor homing peptide, CLSGSLSC (SEQ ID NO: 4), which was identified by in vivo panning of a melanoma (see Example VIII, below).

These results demonstrate that tumor homing peptides selectively home to tumors, particularly to the vasculature in the tumors and that tumor homing peptides identified, for example, by in vivo panning against a breast carcinoma also selectively home to other tumors, including Kaposi's sarcoma and melanoma. In addition, these results demonstrate that immunohistochemical analysis provides a convenient assay for identifying the localization of phage expressing tumor homing peptides.

### EXAMPLE VIII

### Identification of Tumor Homing Peptides by in vivo Panning against a Melanoma Tumor

The general applicability of the in vivo panning method to identify tumor homing peptides was examined by performing in vivo panning against an implanted mouse melanoma tumor.

Mice bearing a melanoma were produced by implantation of B16B15b mouse melanoma cells, which produce highly vascularized tumors. B16B15b mouse melanoma cells were injected subcutaneously into the mammary fat pad of nude mice (2 months old) and tumors were allowed to grow until the diameter was about 1 cm. in vivo panning was performed as disclosed above. Approximately 1×10¹² transducing units of phage expressing the CX₅C (SEQ ID NO: 9), CX₆C (SEQ ID NO: 10) or CX₇C (SEQ ID NO: 11) library were injected, iv, and allowed to circulate for 4 min. Mice then were snap frozen in liquid nitrogen or perfused through the heart while under anesthesia, tumor tissue and brain (control organ) were removed, and phage were isolated as described above. Three rounds of in vivo panning were performed.

TABLE 3
PEPTIDES FROM PHAGE RECOVERED
FROM MOUSE B16B15b MELANOMA

| | | |
|---|---|---|
| CLSGSLSC (4*) | GICKDDWCQ (107) | TSCDPSLCE (103) |
| KGCGTRQCW (109) | YRCREVLCQ (110) | CWGTGLC (111) |
| WSCADRTCM (112) | AGCRLKSCA (113) | SRCKTGLCQ (114) |
| PICEVSRCW (115) | WTCRASWCS (116) | GRCLLMQCR (117) |
| TECDMSRCM (118) | ARCRVDPCV (119) | CTEGVLGGC (120) |
| CSVANSC (121) | CSSTMRC (122) | SIOSTTF (123) |
| GPSRVGG (124) | WWSGLEA (125) | LGTDVRQ (126) |
| LVGVRLL (127) | GRPGDIW (128) | TVWNPVG (129) |
| GLLLVVP (130) | FAATSAE (131) | WCCRQFN (132) |
| VCFGKAL (133) | DSSLRLP (134) | KLWCAMS (135) |
| SLVSFLG (136) | GSFAFLV (137) | LASVRWA (138) |
| TWGHLRA (139) | QYREGLV (140) | QSADRSV (141) |
| YMFWTSR (142) | LVRRWYL (143) | TARGSSR (144) |
| TTREKNL (145) | PKWLLFS (146) | LRTNVVH (147) |
| AVMGLAA (148) | VRNSLRN (149) | |

* - numbers in parentheses indicate SEQ ID NO:.

The amino acid sequences were determined for the inserts in 89 cloned phage recovered from the B16B15b tumors. The peptides expressed by these phage were represented by two predominant sequences, CLSGSLSC (SEQ ID NO: 4; 52% of the clones sequenced) and WGTGLC (SEQ ID NO: 18; 25% of the clones; see Table 3). Reinfection of phage expressing one of the selected peptides resulted in approximately three-fold enrichment of phage homing to the tumor relative to brain.

Localization of the phage expressing a tumor homing peptide in the mouse organs also was examined by immunohistochemical staining of the tumor and various other tissues (see Example VII). In these experiments, 1×10⁹ pfu of a control (insertless) phage or a phage expressing the tumor homing peptide, CLSGSLSC (SEQ ID NO: 4), were injected, iv, into tumor bearing mice and allowed to circulate for 4 min.

Immunostaining was evident in the melanoma obtained from a mouse injected with phage expressing the CLSGSLSC (SEQ ID NO: 4) tumor homing peptide. Staining of the melanoma generally was localized to the blood vessels within the tumor, although some staining also was present in the tumor parenchyma. Essentially no staining was observed in a tumor obtained from a mouse injected with the insertless control phage or in skin or in kidney samples obtained from mice injected with either phage. However, immunostaining was detected in the liver sinusoids and in spleen, indicating that phage can be trapped nonspecifically in organs containing RES.

Using similar methods, in vivo panning was performed in mice bearing a SLK human Kaposi's sarcoma. Tumor homing peptides were identified and are disclosed in Table 4. Together, these results demonstrate that the in vivo panning method is a generally applicable method for screening a phage library to identify phage expressing tumor homing peptides.

TABLE 4
PEPTIDES FROM PHAGE RECOVERED
FROM HUMAN KAPOSI'S SARCOMA

| | | |
|---|---|---|
| TDCTPSRCT (150*) | SWCQFEKCL (151) | VPCRFKQCW (152) |
| CTAMRNTDC (153) | CRESLKNC (154) | CMEMGVKC (155) |
| VTCRSLMCQ (156) | CNNVGSYC (157) | CGTRVDHC (158) |
| CISLDRSC (159) | CAMVSMED (160) | CYLGVSNC (161) |
| CYLVNVDC (162) | CIRSAVSC (163) | LVCLPPSCE (164) |
| RHCFSQWCS (165) | FYCPGVCCR (166) | ISCAVDACL (167) |
| EACEMAGCL (168) | PRCESQLCP (169) | RSCTKHQCP (170) |
| QWCSRRWCT (171) | MFCRMRSCD (172) | GICKDLWCQ (173) |
| NACESATCG (174) | APCGLLACI (175) | NRCRGVSCT (176) |
| FPCEGKKCL (177) | ADCRQKPCL (178) | FGCVMASCR (179) |
| AGCINGLCG (180) | RSCAEPWCY (181) | DTCRALRCN (182) |
| KCCGTRQCW (109) | GRCVDGGCT (183) | YRCIARECE (184) |
| KRCSSSLCA (185) | ICLLAHCA (186) | QACPMLLCM (187) |
| LDCLSELCS (188) | AGCRVESC (189) | HTCLVALCA (190) |
| TYCPGQECE (191) | RLCSLYGCV (192) | RKCEVPGCQ (193) |
| EDCTSRFCS (194) | LECVVDSCR (195) | EICVDGLCV (196) |
| RWCREKSCW (197) | FRCLERVCT (198) | RPCGDQACE (199) |
| CNKTDODEGVTC (15) | | |

* - numbers in parentheses indicate SEQ ID NO:.

### EXAMPLE IX

### Characterization of a Chimeric Peptide Composed of a Prostate-homing Peptide and _{D}(KLAKLAK)₂

This example demonstrates that the chimeric peptide SMSIARL-GG-_{D}(KLAKLAK)₂ can selectively induce apoptosis in prostate tissue following systemic administration and can prolong survival of animals with experimental prostate cancer.

A. Isolation of Prostate Homing Peptides

An X₇ library was injected into mice, and sequences isolated that were preferentially found in prostate as compared to brain as described in WO 99/46284. The prostate homing peptides SMSIARL (SEQ ID NO: 207) and VSFLEYR (SEQ ID NO: 222) exhibited a 34-fold and 17-fold enrichment, respectively, in prostate as compared to brain. Additional prostate homing sequences identified by in vivo panning are shown in Table 5.

TABLE 5
PEPTIDES FROM PI-TAGE RECOVERED FROM PROSTATE

| | |
|---|---|
| EVQSAKW (209) | KRVYVLO (210) |
| GRLSVQV (211) | WKPASLS (212) |
| FAVRVVG (213) | LVRPLEO (214) |
| GFYRMLG (215) | EGRPMVY (216) |
| GSRSLGA (217) | RVWQGDV (218) |
| GOELLA (219) | FVWLVGS (220) |
| GSEPMFR (221) | VSFLEYR (222) |
| WHQPL (223) | SMSIARL* (207) |
| RGRWLAL* (224) | QVEEFPC (225) |
| LWLSGNW (226) | GPMLSVM (227) |
| WTFLERL (228) | VLPGGQW (229) |
| REVKES (230) | RTPAAVM (231) |
| CEWLGEC (232) | PNPLMBL (233) |
| SLWYLGA (234) | YVGGWEL (235) |

Parentheses contain SEQ ID NO:.
*indicates sequences isolated more than once.

B. Prostate Homing of Prostate-homing Peptide Biotin Conjugates

Using in vivo screening of a heptapeptide phage library, a prostate-homing peptide has been identified that concentrates in the prostate 35-fold more than in various other tissues. This phage displays the peptide SMSIARL (SEQ ID NO: 207). Coinjecting synthetic SMSIARL peptide (SEQ ID NO: 207) inhibited the prostate-selective homing of phage bearing SMSIARL (SEQ ID NO: 207). Furthermore, antibody staining of tissue sections showed that SMSIARL (SEQ ID NO: 207) phage localized to prostate tissue, but not to other tissues after an intravenous injection of the phage into mice. Control phage also did not accumulate in the prostate. SMSIARL (SEQ ID NO: 207) phage also homed to rat prostate tissue.

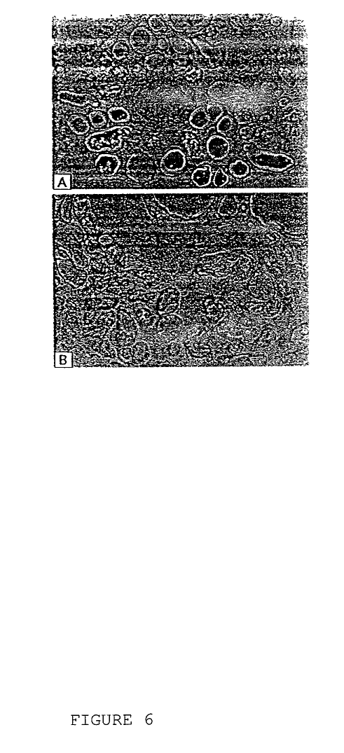

As shown in FIG. 6, the biotin-conjugated SMSIARL (SEQ ID NO: 207) synthetic peptide was shown to home to the prostate. Briefly, one mg of biotin-conjugated prostate-homing peptide SMSIARL (SEQ ID NO: 207), or biotin-labeled control peptide CARAC (SEQ ID NO: 208), was injected intravenously into a mouse, which was sacrificed 10 minutes later. The prostate and other tissues were collected, sectioned and processed for staining with avidin-peroxidase. Biotin staining was found mostly in the lumen of the glands, rather than in the vasculature, as soon as 10 minutes after systemic injection of the conjugate. These results indicate that the SMSIARL (SEQ ID NO: 207) peptide, as well as other moieties attached to it such as phage or biotin, translocates to the prostate epithelium and then into the glandular lumen.

C. Induction of Prostate-selective Apoptosis

The ability of the SMSIARL peptide (SEQ ID NO: 207) to deliver a pro-apoptotic peptide, _{D}(KLAKLAK)₂, to the prostate was analyzed. Briefly, SMSIARL-GG-_{D}(KLAKLAK)₂ chimeric peptide or control material was administered as a single dose of 250 μg peptide/mouse, and TUNEL staining was performed on tissues obtained 24 hours later. As shown in FIG. 7, mice injected with the SMSIARL-GG-_{D}(KLAKLAK)₂ chimera showed increased apoptosis in their prostates and, in particular, in capillary endothelium and the basal myoepithelial cells of prostate glands. There was no evidence of increased apoptosis in other tissues such as the testis, kidney or brain. Apoptosis was not observed in negative control mice treated with 250 μg of an unconjugated mixture of SMSIARL (SEQ ID NO: 207) and _{D}(KLAKLAK)₂.

D. SMSIARL-GG-_{D}(KLAKLAK)₂ Treatment of TRAMP Mice

TRAMP mice develop prostate cancer under the influence of a transgene as described in Gingrich et al., supra, 1996. SMSIARL-GG-_{D}(KLAKLAK)₂ chimeric peptide was assayed for the ability to suppress cancer development in the TRAMP mice. Treatment was initiated at 12 weeks of age with mice (10 per group) receiving SMSIARL-GG-_{D}(KLAKLAK)₂ peptide or control peptide at 250 μg/dose every other week for a total of 10 doses. Four mice, which had no visible tumor, were eliminated from the SMSIARL-GG-_{D}(KLAKLAK)₂ group after dying within minutes after the injection. As shown in FIG. 8, SMSIARL-GG-_{D}(KLAKLAK)₂-treated mice survived longer than mice treated with controls, which were vehicle, _{D}(KLAKLAK)₂ peptide alone, or SMSIARL peptide (SEQ ID NO: 207) alone. Thus, treatment of TRAMP mice with targeted pro-apoptotic SMSIARL-GG-_{D}(KLAKLAK)₂ compound over a period of several months resulted in an apparent increase in survival of the treated mice relative to control mice.

E. Prostate-homing SMSIARL (SEO ID NO: 207) Phage Bind to Human Prostate Vasculature

A human prostate tissue section containing both normal and cancerous tissue was overlaid with 10⁹ TU SMSIARL phage (SEQ ID NO: 207), and the binding of the phage was detected with anti-phage antibody and peroxidase staining. As shown in FIG. 9, SMSIARL (SEQ ID NO: 207) phage bind to the endothelium of human prostate blood vessels (see panels a and b). No endothelial staining was seen with phage that contain no peptide insert (panel c). Furthermore, the SMSIARL (SEQ ID NO: 207)-phage staining was inhibited when soluble SMSIARL peptide SEQ ID NO: 207 was included in the overlay at 0.3 mg/ml. The results shown in FIG. 9 indicate that at least some tumors retain the receptor for the homing peptide. Furthermore, peptide SEQ ID NO: 207 can bind to vessels in intraprostatic cancer, while blood vessels in several other human tissues were not stained by the SMSIARL (SEQ ID NO: 207) phage.

All journal article, reference, and patent citations provided above, in parentheses or otherwise, whether previously stated or not, are incorporated herein by reference.

Although the invention has been described with reference to the examples above, it should be understood that various modifications can be made without departing from the spirit of the invention. Accordingly, the invention is limited only by the following claims.

We claim:

1. A chimeric prostate-homing pro-apoptotic peptide, comprising a prostate-homing peptide linked to an antimicrobial peptide,
said chimeric peptide selectively internalized by prostate tissue and exhibiting high toxicity thereto, and
said antimicrobial peptide having low mammalian cell toxicity when not linked to said prostate-homing peptide.

2. The chimeric peptide of claim 1, wherein said prostate-homing peptide comprises the sequence SMSIARL (SEQ ID NO: 207), or a functionally equivalent sequence.

3. The chimeric peptide of claim 1, wherein said antimicrobial peptide has an amphipathic α-helical structure.

4. The chimeric peptide of claim 1, wherein said antimicrobial peptide comprises a sequence selected from the group consisting of:
(KLAKLAK)₂ (SEQ ID NO: 200);
(KLAKKLA)₂ (SEQ ID NO: 201);
(KAAKKAA)₂ (SEQ ID NO: 202); and
(KLGKKLG)₃ (SEQ ID NO: 203).

5. The chimeric peptide of claim 1, wherein said antimicrobial peptide comprises the sequence _{D}(KLAKLAK)₂.

6. The chimeric peptide of claim 5, comprising the sequence SMSIARL-GG-_{D}(KLAKLAK)₂.

7. The chimeric peptide of claim 6, consisting of the sequence SMSIARL-GG-_{D}(KLAKLAK)₂.

8. A method of directing an antimicrobial peptide in vivo to a prostate cancer, comprising administering the chimeric peptide of claim 1.

9. The method of claim 8, wherein said prostate-homing peptide comprises the sequence SMSIARL (SEQ ID NO: 207), or a functionally equivalent sequence.

10. The method of claim 8, wherein said antimicrobial peptide comprises the sequence _{D}(KLAKLAK)₂.

11. The method of claim 10, wherein said chimeric peptide comprises the sequence SMSIARL-GG-_{D}(KLAKLAK)₂.

12. The method of claim 11, wherein said chimeric peptide is SMSIARL-GG-_{D}(KLAKLAK)₂.

13. A method of inducing selective toxicity in vivo in a prostate cancer, comprising administering the chimeric peptide of claim 1 to a subject having prostate cancer.

14. The method of claim 13, wherein said prostate-homing peptide comprises the sequence SMSIARL (SEQ ID NO: 207), or a functionally equivalent sequence.

15. The method of claim 13, wherein said antimicrobial peptide comprises the sequence _{D}(KLAKLAK)₂.

16. The method of claim 15, wherein said chimeric peptide comprises the sequence SMSIARL-GG-_{D}(KLAKLAK)₂.

17. The method of claim 16, wherein said chimeric peptide is SMSIARL-GG-_{D}(KLAKLAK)₂.

18. A method of treating a patient having a prostate cancer, comprising administering the chimeric peptide of claim 1 to said patient, whereby said chimeric peptide is selectively toxic to said tumor.

19. The method of claim 18, wherein said prostate-homing peptide comprises the sequence SMSIARL (SEQ ID NO: 207), or a functionally equivalent sequence.

20. The method of claim 18, wherein said antimicrobial peptide comprises the sequence _{D}(KLAKLAK)₂.

21. The method of claim 20, wherein said chimeric peptide comprises the sequence SMSIARL-GG-_{D}(KLAKLAK)₂.

22. The method of claim 21, wherein said chimeric peptide is SMSIARL-GG-_{D}(KLAKLAK)₂.
